(19) United States
(12) Patent Application Publication
(10) Pub. No.: US 2025/0049963 A1
(43) Pub. Date: Feb. 13, 2025

(54) GLYCOSYLATED DENDRIMERS FOR TARGETED INTRACELLULAR DELIVERY

(71) Applicant: The Johns Hopkins University, Baltimore, MD (US)

(72) Inventors: Kannan Rangaramanujam, Highland, MD (US); Rishi Sharma, Baltimore, MD (US); Anjali Sharma, Fallston, MD (US); Kevin Liaw, Baltimore, MD (US); Sujatha Kannan, Highland, MD (US)

(21) Appl. No.: 18/722,515

(22) PCT Filed: Dec. 20, 2022
(86) PCT No.: PCT/US2022/082033

§ 371 Date: Jun. 20, 2024

Related U.S. Application Data
(60) Provisional application No. 63/291,486, filed on Dec. 20, 2021.

(51) Int. Cl. A61K 49/00; A61K 47/59

(52) U.S. Cl. CPC A61K 49/0054; A61K 47/595; A61K 49/0032

(57) ABSTRACT

Conjugation of dendrimer molecules with one or more carbohydrate moieties significantly enhances targeting of tumor-associated macrophages (TAMs) and microglia by increasing brain penetration and cellular internalization, as compared with dendrimers without modification with carbohydrate molecules. Compositions of dendrimers conjugated with carbohydrate moieties, particularly glucose and/or glucosamine, and one or more active agents to prevent, treat, or diagnose a disease or disorder in a subject in need thereof, and methods of use thereof, have been developed. The compositions are particularly suited for treating and/or ameliorating brain and/or CNS tumors. Methods of treating a human subject having or at risk of brain and/or CNS tumors are provided.

### CROSS-REFERENCE TO RELATED APPLICATIONS

This application claims the benefit of and priority to U.S. Provisional Application No. 63/291,486 filed Dec. 20, 2021, which is hereby incorporated by reference in its entirety.

### STATEMENT REGARDING FEDERALLY SPONSORED RESEARCH

This invention was made with government support under grant RO1 EY025304 and RO1 NS093416 awarded by the National Institutes of Health. The government has certain rights in the invention.

### FIELD OF THE INVENTION

The invention is generally in the field of drug delivery and, in particular, compositions and methods for delivering drugs selectively to macrophage cells within a tumor via dendrimer formulations.

### BACKGROUND OF THE INVENTION

Glioblastoma is among the most aggressive forms of cancers, with a median survival of between 15 and 20 months with maximum clinical intervention. Glioblastomas account for 70% of brain cancer cases, with more than 14,000 new cases diagnosed in the United States each year (Omuro et al., JAMA, 310 (2013) 1842-1850). In addition to these poor prognoses, glioblastoma also exerts significant impacts on the quality of life and cognitive functions of patients.

Current standard of care includes maximum safe surgical resection followed by intensive chemotherapy and radiotherapy, but this intervention strategy has failed to significantly improve patient outcomes. Therefore, innovative strategies to enhance drug delivery to glioblastoma are necessary to achieve robust improvements to patient outcomes. (Becker et al., The Cancer Journal, 18 (2012) 12-19.)

For systemic therapies for glioblastoma to be effective, they must penetrate the blood-brain-tumor barrier. In addition, they must then access and accumulate within target cells at relevant therapeutic doses to achieve efficacy. Tumor-associated macrophages (TAMs) have emerged as promising cellular targets to enable effective tumor therapy due to their roles in regulating the tumor immune response. (Solinas, et al., J. Leukocyte Biol., 86 (2009) 1065-1073; Sica, et al., Eur. J, of Cancer, 42 (2006) 717-727.) Tumors secrete immune polarizing signals that recruit host macrophage and resident microglia and repolarize them into TAMs, which suppress tumor-killing immune activation and promote tumor growth, invasion, and drug resistance. (Jeong, et al., Cancer Research, 79 (2019) 795; Lin, et al., J. of Hem. & Oncol., 12 (2019) 76.)

Based on strong preclinical results, it is believed that targeting TAMs with immunotherapies can reprogram the tumor immune response away from the tumor-supporting state and towards the anti-tumor phenotype. Multiple TAMs-targeted immunotherapies are undergoing clinical trials as single therapies or in combination with traditional treatments in many types of cancers, including glioblastoma (see, for example, clinical trials #NCT02829723, NCT02452424, NCT01349049, and NCT01217229). However, translation has been limited by low response rates, drug resistance, and off-target immune activation leading to systemic toxicities.

Nanotechnology-mediated immunotherapies that specifically target TAMs can provide significant innovation for improved therapeutic efficacy and limited systemic toxicities. Peptide and small molecule targeting ligands can be attached to nanoparticles to improve tumor and cell-specific targeting. (Sun, et al., Angewandte Chemie (International ed. in English), 53 (2014) 12320-12364.) Similar strategies have been employed to target TAMs as well by leveraging overexpression of receptors consistent with the largely anti-inflammatory, pro-tumor phenotype. (Cieslewicz, et al., Proceedings of the National Academy of Sciences, 110 (2013) 15919; Conde, et al., Advanced Functional Materials, 25 (2015) 4183-4194.)

Sugar moieties have recently been revealed as promising targeting ligands to bring therapies to cancer cells and TAMs, which take advantage of the increased metabolism in the tumor (Azad, et al., J Cytol Mol Biol, 1 (2014) 1000003; Calvaresi, et al., Chemical science, 4 (2013) 2319-2333. Sztandera, et al., Pharmaceutical Research, 36 (2019) 140; Zhao, et al., Chemical science, 9 (2018) 2674-2689; Zhao, et al., Theranostics, 10 (2020) 1355-1372). These strategies target altered metabolism and receptor expression in cancer cells and TAMs to achieve drug delivery. In addition, sugars are highly water soluble and nontoxic, facilitating formulation and translation.

Dendrimers have emerged as promising vehicles for targeted drug and gene delivery, and dendrimer-based nanomedicines have shown great potential in targeting neuroinflammation and brain tumors. (Lesniak, et al., Molecular Pharmaceutics, 10 (2013) 4560-4571; Zhang, et al., Biomaterials, 52 (2015); Srinageshwar, et al., International Journal of Molecular Sciences, 18 (2017) 628; Sharma, et al., Science Advances, 6 (2020) eaay8514; Sharma, et al., Journal of Controlled Release, 323 (2020) 361-375; Sharma, et al., Bioengineering & Translational Medicine, 3 (2018) 87-101; Nemeth, et al., Nanoscale, 12 (2020) 16063-16068; Mignani, et al., New Journal of Chemistry, 37 (2013) 3337-3357; Ban, et al., Macromolecular Bioscience, 2000392; and Liu, et al., Nanotheranostics, 3 (2019) 311-330.)

Dendrimers are multivalent yet monodisperse and precise macromolecules forming a nanoplatform for targeted drug delivery systems. Polyamidoamine (PAMAM) generation 4 hydroxyl-terminated dendrimers are able to cross the impaired blood-brain barrier upon systemic administration and selectively target activated microglia/macrophages in a variety of neurodegenerative diseases. (Nance, et al., Journal of Neuroinflammation, 14 (2017) 252; Sharma, et al., Journal of Controlled Release, 283 (2018) 175-189; Zhang, et al., Journal of controlled release, 249 (2017) 173-182.) It has also been shown in the context of cerebral palsy that mannose-modified dendrimers can alter the internalization pathway of dendrimers in activated glia.

However, at present, the available anti-cancer therapies fail to provide effective interventions for glioblastoma and other proliferative diseases in the brain, due to difficulty in penetrating into the affected tissue, and severe toxicities in surrounding healthy tissues. New systems for the targeted delivery of therapeutic anti-cancer agents selectively to target cells within brain tumors are needed.

Therefore, it is an object of the invention to provide compositions that selectively target active agents to target cells within brain tumors, and methods of making and using thereof.

It is also an object of the invention to provide compositions for the treatment or prevention of one or more symptoms of glioblastomas through modulation of the tumor micro-environment.

### SUMMARY OF THE INVENTION

It has been established that conjugation of dendrimer molecules with carbohydrate moieties significantly enhances targeting of tumor-associated macrophages (TAMs) and microglia by increasing brain penetration and cellular internalization, as compared with modification with other types of molecules. Conjugation of dendrimers with carbohydrate moieties such as glucose molecules drives selective uptake of the dendrimers by TAMs in vivo. Compositions and methods of making and using glycosylated dendrimers complexed to, covalently conjugated to, or having intra-molecularly dispersed or encapsulated therein one or more therapeutic or prophylactic agents for treating or preventing brain cancers have been developed.

Glycosylated dendrimer complexes, including a generation 0-10 dendrimer, one or more carbohydrate moieties and one or more active agents, have been synthesized and tested. The one or more carbohydrate moieties and the one or more active agents are conjugated to terminal groups on the surface of the dendrimer, optionally via a linker or spacer. The carbohydrate moiety is typically conjugated to between about 1% and about 25%, inclusive, of the total number of terminal groups on the dendrimer. Preferably, the dendrimer is a hydroxyl (OH)-terminated dendrimer. Exemplary dendrimers include generation 2, generation 3, generation 4, generation 5, generation 6, generation 7, or generation 8 dendrimer. In a preferred embodiment, the dendrimer is a poly(amidoamine) (PAMAM) dendrimer. The carbohydrate moieties are typically present in an amount between about 5% to 15%, inclusive, of the mass of the dendrimer.

Generally, the carbohydrate moieties are those that can be transported via one or more glucose transporters selected from the group consisting of GLUT1, GLUT2, GLUT3, GLUT4, GLUT5, GLUT6, GLUT7, GLUT8, GLUT9, GLUT10, GLUT11, GLUT12, GLUT13, and GLUT14. In preferred embodiments, the carbohydrate moieties are those that can be transported via GLUT1. In some embodiments, the carbohydrate moieties are oligosaccharides with terminal groups selected from glucose, glucosamine, mannose, fructose, dehydroascorbic acid, urate, and myo-inositol. In other embodiments, the carbohydrate moieties are monosaccharides selected from glucose, glucosamine, mannose, fructose, dehydroascorbic acid, urate, and myo-inositol. In preferred embodiments, the carbohydrate moieties are one or more of glucose and/or glucosamine molecules. In further embodiments, the carbohydrate moieties are not galactose.

The one or more active agents is generally selected from a therapeutic agent, a prophylactic agent, and a diagnostic agent. Exemplary active agents include a small molecule, an antibody or antigen-binding fragment thereof, a nucleic acid, and a polypeptide. Exemplary therapeutic agents include anti-cancer agents, immune-modulatory agents, antimicrobial agents, anesthetic agents, antioxidant agents and anti-angiogenic agents. Exemplary diagnostic agents include fluorescent dyes, near infra-red dyes, SPECT imaging agents, PET imaging agents and radioisotopes.

In some embodiments, the glycosylated dendrimers include one or more linkers or coupling agents between the dendrimer and the active agent(s), or between the dendrimer and glucose molecule(s). In further embodiments, the glycosylated dendrimers include one or more polymers bound to an outer surface of the dendrimer, or active agent. For example, in some embodiments, the glycosylated dendrimers comprise oligoethylene glycol chains.

Pharmaceutical formulations of glycosylated dendrimers, including a generation 0-10 dendrimer, one or more carbohydrate moieties and one or more active agents; and a pharmaceutically acceptable carrier or excipient are also described. In some embodiments, the formulation is formulated for intravenous or intraperitoneal administration. In other embodiments, the formulation is formulated for oral administration.

Methods for treating or preventing one or more symptoms of a disease and/or disorder in a subject in need thereof are also described. Typically, the methods administer a pharmaceutical formulation of glycosylated dendrimers, including a generation 0-10 dendrimer, one or more carbohydrate molecules, and one or more therapeutic or prophylactic agents; and a pharmaceutically acceptable carrier or excipient to the subject in an amount effective to treat, alleviate or prevent one or more symptoms of the disease and/or disorder. In some embodiments, the methods treat or prevent one or more proliferative diseases. A preferred proliferative disease is cancer, such as a cancer of the brain and/or CNS. Exemplary brain or CNS cancers that can be treated or prevented according to the methods include gliomas, glioblastoma multiforme, gliosarcoma, astrocytoma, oligodendroglioma, ependymoma or intracranial ependymoblastoma, meningioma, medulloblastoma, ganglioma, head and neck squamous cell carcinoma, Schwannoma, craniopharyngioma, cordomas and pituitary tumor. In some embodiments, the methods reduce or prevent one or more symptoms of the brain cancer or CNS cancer, including headaches, seizures (fits), persistently feeling sick (nausea), being sick (vomiting) and drowsiness, mental or behavioral changes, such as memory problems or changes in personality, progressive weakness, or paralysis on one side of the body, and vision or speech problems. Typically, the amount of active agent effective to treat or prevent the one or more symptoms when conjugated to glycosylated dendrimers is less than the amount of the same active agent administered alone, or administered as a formulation in combination with dendrimers in the absence of associated carbohydrate moieties. For example, in some embodiments, the amount of active agent effective to treat or prevent the one or more symptoms when conjugated to glycosylated dendrimers is at least 10-fold less than the amount of the same active agent administered alone, or administered as a formulation in combination with dendrimers in the absence of associated carbohydrate moieties.

Methods for labeling a tumor in a subject are also provided. Typically, the methods administer a pharmaceutical formulation of glycosylated dendrimers, including a generation 0-10 dendrimer, one or more glucose molecules and one or more diagnostic agents; and a pharmaceutically acceptable carrier or excipient to the subject in an amount effective to label one or more cells associated with the tumor in the subject. In some embodiments the labeling is used to diagnose or identify a tumor in the subject. For example, in some embodiments the labeling is used to monitor or guide chemotherapy and/or surgery.

The formulation is generally administered to the subject systemically. In some embodiments, the formulation is administered via the intravenous or intraperitoneal route. In other embodiments, the formulation is administered via oral administration. In some embodiments, the formulation is administered prior to, in conjunction, subsequent to, or in alternation with treatment with one or more additional therapies or procedures. Exemplary additional procedures include administering one or more therapeutic, prophylactic and/or diagnostic agents or radiation therapy.

Methods of complexing and/or conjugating dendrimers with a plurality of surface glucose moieties to one or more therapeutic, prophylactic, and/or diagnostic agents are described. Three glycodendrimers were synthesized and tested for targeting tumor and TAMs from systemic administration: a) glucosylated-PAMAM-OH, b) galactosylated PAMAM-OH, and mannosylated-PAMAM-OH. Glucose modification significantly enhanced targeting of TAMs by increasing brain penetration and cellular internalization, while galactose modification shifts targeting away from TAMs towards galectins on glioblastoma tumor cells. Mannose modification did not alter TAMs and microglia targeting of these dendrimers but did alter their kinetics of accumulation within the GBM tumor. The whole body biodistribution was largely similar between the systems. These glycosylated dendrimers are powerful platforms for the treatment of glioblastoma and other cancers, showing >100-fold TAM specificity compared to traditional ligand-targeted nanoparticle platforms. The examples demonstrate that the glucose-conjugated dendrimer nanoparticles do not target cancer cells but rather, target tumor associated macrophages and microglia (TAMs) and enhance their uptake into these cells, >10-fold higher than non-glycosylated PAMAM-OH dendrimers.

### BRIEF DESCRIPTION OF THE DRAWINGS

FIGS. 1A-1B are schematics showing molecular structures in a stepwise synthetic route for producing dendrimer-sugar conjugates. FIG. 1A shows structural representations of clickable sugars, including structures of glucose-azide, galactose-azide, and mannose-azide with PEG linkers (β-D-Glucose-PEG4-azide (la), β-D-Galactose-PEG4-azide (1b), and α-D-Mannose-PEG4-azide (1c)). FIG. 1B shows the synthetic scheme for dendrimer-sugar conjugates. The synthesis of fluorescently labeled Cy5-D-Glucose, Cy5-D-Mannose, and Cy5-D-Galactose is presented using a CuAAC click chemistry approach.

FIGS. 2A-2B are graphs showing how sugar conjugation alters tumor accumulation kinetics of dendrimers and increases tumor specificity. Graphs show Dendrimer (0-50 μg/g tissue) (FIG. 2A) and Tumor to Con. Hemisphere Ratio (0-30) (FIG. 2B) over time post administration (1, 4, and 24 hrs) for each of unmodified dendrimers (D-OH), glucose-(D-GLU), mannose-(D-MAN), and galactose-conjugated dendrimers (D-GAL), respectively. ** p<0.01, *** p<0.001.

FIG. 3 is a bar graph of in vitro dendrimer uptake in GL261 cells, showing concentrations of Dendrimer (0-0.35 pg/cell) for each of unmodified dendrimers (D-OH), glucose-(D-GLU), mannose-(D-MAN), and galactose-conjugated dendrimers (D-GAL), respectively.

FIGS. 4A-4E are graphs showing systemic biodistribution of sugar-conjugated dendrimers compared to unmodified dendrimers. Glioblastoma-bearing mice were injected with unmodified dendrimers (D-OH), glucose-(D-GLU), mannose-(D-MAN), or galactose-conjugated dendrimers (D-GAL) shows dendrimer in plasma (0-10% ID/ml plasma) over time (1-24 hours after injection) (FIG. 4A); dendrimer in kidneys (0-8% ID) (FIG. 4B); dendrimer in liver (0-15% ID) (FIG. 4C); dendrimer in spleen (0-0.6% ID/ml plasma) (FIG. 4D); dendrimer in lungs (0-1.5% ID) (FIG. 4E); and dendrimer in heart (0-1.0% ID) (FIG. 4F) for each of unmodified (D-OH), glucose-(D-GLU), mannose-(D-MAN), and galactose-conjugated (D-GAL) dendrimers, respectively, on day 14 after tumor inoculation. All dendrimers rapidly clear from the body, with less than 1% of the initial injected dose remaining per mL of plasma after 24 hours. Conjugation with sugars alters systemic biodistribution. * p<0.05, ** p<0.01, *** p<0.001.

FIG. 5 is a bar graph of cytotoxicity in sugar-conjugated dendrimers in BV2 murine microglia, showing cell viability (0-125% of control), over treatment with varying concentrations (0.01-1000 μg/ml) of each of glucose-(D-GLU), mannose-(D-MAN), and galactose-conjugated (D-GAL), respectively dendrimers for 24 hours. D-GLU and D-MAN did not impact cell viability in the concentration range tested. D-GAL shows slight cytotoxicity at high dose.

FIGS. 6A-6E are graphs showing how dendrimer-glucose and dendrimer-galactose conjugates alter cellular interactions. FIG. 6A shows dendrimer (0.5 to 1.1-fold change) for each of glucose-conjugated dendrimers (D-GLU) and unmodified (D-OH) dendrimers, respectively, with (+STF-31) or without (−STF-31) blocking GLUT-1 receptors on BV2 murine microglia to validate the GLUT-1 uptake mechanism of D-GLU. ** p<0.01D, n.s. p>0.1. FIG. 6B shows dendrimer (0-1.0 pg/cell) internalization for each of glucose-conjugated dendrimers (D-GLU) and unmodified (D-OH) dendrimers, respectively, with (+IL4) or without (−IL4) in resting microglia significantly greater with D-GLU than D-OH, indicating improved cellular internalization. * p<0.05, *** p<0.001. FIG. 6C shows dendrimer (0-1.0 μg/g tissue) internalization for each of glucose-conjugated dendrimers (D-GLU) and unmodified (D-OH) dendrimers, respectively, in healthy brain tissue (Healthy) and in the contralateral hemisphere (C.H.), respectively, showing similar brain uptake of tumor-bearing brains and significantly greater than D-OH, indicating increased blood brain barrier penetration. ** p<0.01, n.s. p>0.1. FIG. 6D shows Dendrimer (0-0.15 μg/mg protein) internalization for each of dendrimer-galactose (D-GAL) and unmodified (D-OH) dendrimers, respectively, in the presence (+α-lactose) and absence (−α-lactose) of α-lactose, validating D-GAL interactions with galectin surface receptors. * p<0.05, ** p<0.01. FIG. 6E shows dendrimer (0-0.10 μg/mg protein) cellular uptake for each of dendrimer-galactose (D-GAL), and unmodified (D-OH) dendrimers, respectively in the presence (+α-lactose) and absence (−α-lactose) of α-lactose, respectively, showing D-GAL is not altered with α-lactose treatment, indicating that D-GAL interactions with galectin receptors does not impact cellular internalization. n.s. p>0.1.

### DETAILED DESCRIPTION OF THE INVENTION

### I. Definitions

The terms “active agent” or “biologically active agent” are used interchangeably to refer to a chemical or biological compound that induces a desired pharmacological and/or physiological effect, which may be prophylactic, therapeutic, or diagnostic. These may be a nucleic acid, a nucleic acid analog, a small molecule having a molecular weight less than 2 kD, more typically less than 1 kD, a peptidomimetic, a protein or peptide, carbohydrate or sugar, lipid, or a combination thereof. The terms also encompass pharmaceutically acceptable, pharmacologically active derivatives of agents, including, but not limited to, salts, esters, amides, prodrugs, active metabolites, and analogs. The term “therapeutic agent” refers to an agent that can be administered to treat one or more symptoms of a disease or disorder. The term “diagnostic agent” generally refers to an agent that can be administered to reveal, pinpoint, and define the localization of a pathological process. The diagnostic agents can label target cells that allow subsequent detection or imaging of these labeled target cells. In some embodiments, diagnostic agents can, via dendrimer or suitable delivery vehicles, target/bind activated macrophages and tumor-associated microglia (TAMs). The term “prophylactic agent” generally refers to an agent that can be administered to prevent disease or to prevent certain conditions, such as a vaccine.

The terms “immunologic”, “immunological” or “immune” response is the development of a beneficial humoral (antibody mediated) and/or a cellular (mediated by antigen-specific T cells or their secretion products) response directed against an immunogen in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺T helper cells and/or CD8⁺ cytotoxic T cells. The response may also involve activation of monocytes, macrophages, NK cells, basophils, dendritic cells, astrocytes, microglia cells, eosinophils, or other components of innate immunity. The presence of a cell-mediated immunological response can be determined by proliferation assays (CD4⁺T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The terms “immunomodulatory agent” or “immunotherapeutic agent” refer to an active agent that can be administered to regulate, enhance, reduce, prolong, decrease, or otherwise alter one or more factors of the innate or adaptive immune response in the recipient. Generally, immunomodulatory agents can modulate immune microenvironment for a desired immunological response by targeting one or more immune cells or cell types at a target site, and thus, are not necessarily specific to any cancer type. In some embodiments, the immunomodulatory agents are specifically delivered to inhibit or reduce suppressive immune cells such as tumor associated macrophages for an enhanced anti-tumor response at a tumor site.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more brain cancers or disorders, such as inhibiting or reducing serum levels of one or more markers of brain cancer, such as MGMT promoter methylation, co-deletion of 1p and 19q, IDH1/2 mutations, and other select pathway-associated mutations.

The terms “inhibit” or “reduce” in the context of inhibition, mean to reduce, or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, dendrimer compositions including one or more inhibitors may inhibit or reduce the activity and/or quantity of activated microglia by about 10%, 20%, 30%, 40%, 50%, 75%, 85%, 90%, 95%, or 99% from the activity and/or quantity of the same cells in equivalent tissues of subjects that did not receive, or were not treated with the dendrimer compositions. In some embodiments, the inhibition and reduction are compared at levels of mRNAs, proteins, cells, tissues, and organs. For example, an inhibition and reduction in the size and/or viability of a tumor, or the number or viability of cancer cells in a subject, as compared to an untreated control subject.

The term “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with brain cancer are mitigated or eliminated, including, but are not limited to, reducing serum levels of one or more biomarkers of a brain tumor (e.g., MGMT promoter methylation, co-deletion of 1p and 19q, or IDH1/2 mutations), reducing tumor size, reducing metastasis or metastatic potential, reducing tumor cell viability, reducing long-term morbidity and mortality.

The term “pharmaceutically acceptable salt” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; and N-benzylphenethylamine.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent, or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “biodegradable” generally refers to a material that will degrade or erode under physiologic conditions to smaller units or chemical species that are capable of being metabolized, eliminated, or excreted in vivo. The degradation time is a function of composition and morphology.

The term “dendrimer” includes, but is not limited to, a molecular architecture with an interior core, interior layers, or “generations” of repeating units regularly attached to this initiator core, and an exterior surface of terminal groups attached to the outermost generation.

The term “functionalize” means to modify a compound or molecule in a manner that results in the attachment of a functional group or moiety. For example, a molecule may be functionalized by the introduction of a molecule that makes the molecule a strong nucleophile or strong electrophile.

The term “targeting moiety” refers to a moiety that localizes to or away from a specific location. The moiety may be, for example, a protein, nucleic acid, nucleic acid analog, carbohydrate, or small molecule. The entity may be, for example, a therapeutic compound such as a small molecule, or a diagnostic entity such as a detectable label. The location may be a tissue, a particular cell type, or a subcellular compartment. In one embodiment, the targeting moiety directs the localization of an agent. In preferred embodiment, the dendrimer composition can selectively target activated microglia in the absence of an additional targeting moiety.

The term “prolonged residence time” refers to an increase in the time required for an agent to be cleared from a patient's body, or organ or tissue of that patient. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, 500, 1000, 2000, 5000, or 10000 times longer than a standard of comparison such as a comparable agent without a dendrimer that specifically target specific cell types.

The terms “incorporated” and “encapsulated” refer to incorporating, formulating, or otherwise including an agent into and/or onto a composition that allows for release, such as sustained release, of such agent in the desired application. The agent or other material can be incorporated into a dendrimer, by binding to one or more surface functional groups of such dendrimer (by covalent, ionic, or other binding interaction), by physical admixture, by enveloping the agent within the dendritic structure, and/or by encapsulating the agent inside the dendritic structure.

The term “glycosylated” refers to the presence of one or more carbohydrate moieties conjugated to a structure, such as a dendrimer. For example, the term “glycosylated dendrimer” refers to a dendrimer that is conjugated to one or more carbohydrate molecules such as monosaccharide molecules including glucose, glucosamine, galactose, mannose, and fructose.

The term “glucosylated” refers to the presence of one or more glucose molecules conjugated to a structure, such as a dendrimer. For example, the term “glucosylated dendrimer” refers to a dendrimer that is conjugated to one or more glucose molecules. The term is used to refer to a structure having one or more glucose molecules bound thereto, irrespective of the presence of one or more additional carbohydrates.

### II. Compositions

Dendrimers conjugated or complexed with one or more carbohydrate moieties have greatly increased penetration through brain tissue relative to un-modified dendrimers. Glycosylated dendrimers (dendrimers conjugated with one or more carbohydrate moieties) selectively accumulate within tumor-associated microglial cells (TAMs) within and surrounding tumors, in particular brain tumors.

Compositions of dendrimers conjugated or complexed with one or more carbohydrate moieties suitable for delivering one or more agents, particularly one or more agents to prevent, treat, or diagnose one or more brain cancers and/or diseases, in a subject in need thereof, have been developed. The compositions are particularly suited for treating and/or ameliorating one or more symptoms of glioblastoma in a subject. In preferred embodiments, dendrimers (D) conjugated or complexed with glucose (Glu) are used for delivering one or more agents, particularly one or more agents to prevent, treat, or diagnose one or more brain cancers and/or diseases, in a subject in need thereof.

Preferably, a small proportion, such as 20% or less of the surface groups available at the external surface of the dendrimer are conjugated with glucose. Therefore, preferably the majority, such as 80%, or more than 80%, more than 70%, or more than 60%, of the surface groups remain available for modifications with additional active agents and/or maintained as free, unconjugated terminal hydroxyl groups for the intrinsic brain tumor targeting properties of the dendrimers. In a preferred embodiment, the dendrimer is a hydroxyl-terminated poly(amidoamine) (PAMAM) dendrimer.

Compositions include one or more prophylactic, therapeutic, and/or diagnostic agents encapsulated in, associated with, and/or conjugated to the glycosylated dendrimers. Generally, one or more active agent is encapsulated, associated, and/or conjugated in the dendrimer complex at a concentration of between about 0.01% and about 30% by weight, preferably between about 1% and about 20%, more preferably between about 5% to about 20% by weight. Preferably, an active agent is covalently conjugated to the dendrimer via one or more linkages such as disulfide, ester, ether, thioester, carbamate, carbonate, hydrazine, and amide, optionally via one or more spacers.

The presence of active agents can affect the zeta-potential or the surface charge of the dendrimer-glucose conjugates. In one embodiment, the zeta potential of the dendrimer-glucose conjugated or complexed with active agent(s) is between −100 mV and 100 mV, between −50 mV and 50 mV, between −25 mV and 25 mV, between −20 mV and 20 mV, between −10 mV and 10 mV, between −10 mV and 5 mV, between −5 mV and 5 mV, or between −2 mV and 2 mV. The range above is inclusive of all values from −100 mV to 100 mV. In a preferred embodiment, the surface charge is neutral or near-neutral, i.e., from about −10 mV to about 10 mV, inclusive, preferably from about −1 mV to about 1 mV.

### A. Dendrimers

Dendrimers are three-dimensional, hyperbranched, monodispersed, globular and polyvalent macromolecules including a high density of surface end groups (Tomalia, D. A., et al., Biochemical Society Transactions, 35, 61 (2007); and Sharma, A., et al., ACS Macro Letters, 3, 1079 (2014)). Due to their unique structural and physical features, dendrimers are useful as nanocarriers for various biomedical applications including targeted drug/gene delivery, imaging and diagnosis (Sharma, et al., RSC Advances, 4, 19242 (2014); Caminade, et al., Journal of Materials Chemistry B, 2, 4055 (2014); Esfand, et al., Drug Discovery Today, 6, 427 (2001); and Kannan, et al., Journal of Internal Medicine, 276, 579 (2014)).

Dendrimer surface groups have a significant impact on their biodistribution (Nance, et al., Biomaterials, 101, 96 (2016)). Hydroxyl terminated generation 4 PAMAM dendrimers (˜4 nm size) without any targeting ligand cross the impaired blood/brain barrier (BBB) upon systemic administration in a rabbit model of cerebral palsy (CP) significantly more (>20 fold) as compared to healthy controls, and selectively target activated microglia and astrocytes (Lesniak, et al., Mol Pharm, 10 (2013)).

The term “dendrimer” (“D”) includes a molecular architecture with an interior core and layers, or “generations” of repeating units which are attached to and extend from this interior core, each layer having one or more branching points, and an exterior surface of terminal groups attached to the outermost generation. In some embodiments, dendrimers have regular dendrimeric or “starburst” molecular structures. The dendrimers can have carboxylic, amine, or hydroxyl terminations, and can be of any generation including, but not limited to, generation 1 (“G1”) dendrimers (“D1”), generation 2 (“G2”) dendrimers (“D2”), generation 3 (“G3”) dendrimers (“D3”), generation 4 (“G4”) dendrimers (“D4”), generation 5 (“G5”) dendrimers (“D5”), generation 6 (“G6”) dendrimers (“D6”), generation 7 (“G7”) dendrimers (“D7”), generation 8 (“G8”) dendrimers (“D8”), generation 9 (“G9”) dendrimers (“D9”), or generation 10 (“G10”) dendrimers (“D10”).

Generally, dendrimers have a diameter between about 1 nm and about 50 nm, more preferably between about 1 nm and about 20 nm, between about 1 nm and about 10 nm, or between about 1 nm and about 5 nm. In some embodiments, the diameter is between about 1 nm and about 2 nm, inclusive; between about 2 nm and about 3 nm, inclusive; between about 3 nm and about 5 nm, inclusive; or between about 4 nm and about 5 nm, inclusive. Conjugates are generally in the same size range, although large proteins such as antibodies may increase the size by 5-15 nm. In general, active agents are encapsulated in a weight-to-weight ratio of agent to dendrimer of between 1:1 and 4:1 for the larger generation dendrimers. In preferred embodiments, the dendrimers have a diameter effective to penetrate brain tissue and to retain in target cells for a prolonged period of time.

In some embodiments, dendrimers have a molecular weight between about 500 Daltons and about 100,000 Daltons inclusive, preferably between about 500 Daltons and about 50,000 Daltons inclusive, most preferably between about 1,000 Daltons and about 20,000 Daltons inclusive.

Suitable dendrimer scaffolds that can be used include poly(amidoamine), also known as PAMAM, or STARBURST™ dendrimers, polypropylamine (POPAM), polyethylenimine, polylysine, polyester, iptycene, aliphatic poly(ether), and/or aromatic polyether dendrimers. The dendrimers can have carboxylic, amine and/or hydroxyl terminations. In preferred embodiments, the dendrimers have hydroxyl terminations. Each dendrimer of the dendrimer complex may be same or of similar or different chemical nature than the other dendrimers (e.g., the first dendrimer may include a PAMAM dendrimer, while the second dendrimer may be a POPAM dendrimer).

The term “PAMAM dendrimer” means poly(amidoamine) dendrimer, which may contain different cores, with amidoamine building blocks, and can have carboxylic, amine and hydroxyl terminations of any generation including, but not limited to, generation 1 (G1) PAMAM dendrimers, generation 2 (G2) PAMAM dendrimers, generation 3 (G3) PAMAM dendrimers, generation 4 (G4) PAMAM dendrimers, generation 5 (G5) PAMAM dendrimers, generation 6 (G6) PAMAM dendrimers, generation 7 (G7) PAMAM dendrimers, generation 8 (G8) PAMAM dendrimers, generation 9 (G9)PAMAM dendrimers, or generation 10 (G10) PAMAM dendrimers. In the preferred embodiment, the dendrimers are soluble in the formulation and are generation (“G”) 4, 5 or 6 dendrimers (D4, D5, or D6). The dendrimers may have hydroxyl groups attached to their functional surface groups.

Methods for making dendrimers are known to those of skill in the art and generally involve a two-step iterative reaction sequence that produces concentric shells (generations) of dendritic 0-alanine units around a central initiator core (e.g., ethylenediamine-cores). Each subsequent growth step represents a new “generation” of polymer with a larger molecular diameter, twice the number of reactive surface sites, and approximately double the molecular weight of the preceding generation. Dendrimer scaffolds suitable for use are commercially available in a variety of generations. Preferable, the dendrimer compositions are based on generation 0, 1, 2, 3, 4, 5, 6, 7, 8, 9, or 10 dendrimeric scaffolds. Such scaffolds have, respectively, 4, 8, 16, 32, 64, 128, 256, 512, 1024, 2048, and 4096 reactive sites. Thus, the dendrimeric compounds based on these scaffolds can have up to the corresponding number of combined targeting moieties, if any, and active agents bound or complexed/conjugated thereto.

In some embodiments, the dendrimers include a plurality of hydroxyl (OH) groups. Some exemplary high-density hydroxyl group-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols.

In some embodiments, the high-density hydroxyl groups-containing dendrimers are oligo ethylene glycol (OEG)-like dendrimers. For example, a generation 2 OEG dendrimer (“D2-OH-60”) can be synthesized using highly efficient, robust and atom economical chemical reactions such as Cu (I) catalyzed alkyne-azide click and photo catalyzed thiol-ene click chemistry. Highly dense polyol dendrimers of low generation in minimum reaction steps can be produced by using an orthogonal hypermonomer and hypercore strategy, for example as described in International Patent Publication No. WO 2019/094952. In some embodiments, the dendrimer backbone has non-cleavable polyether bonds throughout the structure to avoid disintegration of dendrimer in vivo and to allow the elimination of such non-biodegradable dendrimers as a single entity from the body.

In some embodiments, the dendrimer specifically targets a particular tissue region and/or cell type following administration into the body. In preferred embodiments, the dendrimer specifically targets a particular tissue region and/or cell type without a targeting moiety.

In preferred embodiments, the dendrimers have a plurality of hydroxyl (—OH) groups on the periphery of the dendrimers. The preferred surface density of hydroxyl (—OH) groups is at least 1 OH group/nm² (number of hydroxyl surface groups/surface area in nm²). For example, in some embodiments, the surface density of hydroxyl groups is more than 2, 3, 4, 5, 6, 7, 8, 9, 10; preferably at least 10, 15, 20, 25, 30, 35, 40, 45, 50, or more than 50. In further embodiments, the surface density of hydroxyl (—OH) groups is between about 1 and about 50, preferably 5-20 OH groups/nm² (number of hydroxyl surface groups/surface area in nm²) while having a molecular weight of between about 500 Da and about 10 kDa.

In some embodiments, the dendrimers may have a fraction of the hydroxyl groups exposed on the outer surface, with the others in the interior core of the dendrimers. In preferred embodiments, the dendrimers have a volumetric density of hydroxyl (—OH) groups of at least 1 OH group/nm³ (number of hydroxyl groups/volume in nm³). For example, in some embodiments, the volumetric density of hydroxyl groups is 2, 3, 4, 5, 6, 7, 8, 9, 10, or more than 10, 15, 20, 25, 30, 35, 40, 45, and 50. In some embodiments, the volumetric density of hydroxyl groups is between about 4 and about 50 groups/nm³, preferably between about 5 and about 30 groups/nm³, more preferably between about 10 and about 20 groups/nm³.

i. Methods of Making Dendrimers

Dendrimers can be prepared via a variety of chemical reaction steps. Dendrimers are usually synthesized according to methods allowing for the control of the dendrimer structure at every stage. The dendrimeric structures are primarily synthesized by one of two different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward, building the dendrimer inwardly, and eventually attaching the structure to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, dendrimer synthesis relies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of agents are linked to one type of dendron and a different type of agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in International Patent Publication Nos. WO 2009/046446, WO 2015168347, WO 2016025745, WO 2016025741, WO 2019094952, and U.S. Pat. No. 8,889,101.

### B. Dendrimers Modified with Carbohydrates

Hydroxyl-terminated dendrimers conjugated with one or more carbohydrate molecules selectively accumulate within activated macrophages and microglial cells and can be used to selectively deliver therapeutic, prophylactic, or diagnostic agents to tumor-associated microglia (TAMs) within the brain. Compositions of hydroxyl-terminated dendrimers modified by addition of one or more carbohydrate moieties to the dendrimer are described. In some embodiments, glycosylated hydroxyl-terminated dendrimers selectively target and internalize in activated macrophages and microglial cells in vitro and in vivo; and/or selectively accumulate within activated macrophages and microglial cells through multivalent binding with glucose receptors, for example, glucose receptors on endothelial cells of the blood brain barrier.

The function of the blood-brain-barrier is necessary to protect and restrict access of toxins and pathogens to the brain and CNS via the bloodstream, but proves a major hindrance to the delivery of drugs within the brain. Hydroxyl-terminated dendrimer nanoparticles overcome the problem of passing across the blood-brain-barrier and penetration into and through the brain tissue, to enable selective accumulation within tumor-associated immune cells at sites of brain tumors, by incorporating ligands to the nanoparticles' surfaces that actively bind with glucose receptors expressed on endothelial cells.

Three glycodendrimers were synthesized and tested for targeting tumor and TAMs from systemic administration: a) glucosylated-PAMAM-OH, b) galactosylated PAMAM-OH, and mannosylated-PAMAM-OH. Glucose modification significantly enhanced targeting of TAMs by increasing brain penetration and cellular internalization, while galactose modification shifts targeting away from TAMs towards galectins on glioblastoma tumor cells. Mannose modification did not alter TAMs and microglia targeting of these dendrimers but did alter their kinetics of accumulation within the GBM tumor. The whole body biodistribution was largely similar between the systems. These glycosylated dendrimers are powerful platforms for the treatment of glioblastoma and other cancers, showing >100-fold TAM specificity compared to traditional ligand-targeted nanoparticle platforms. The examples demonstrate that the glucose-conjugated dendrimer nanoparticles do not target cancer cells, but tumor associated macrophages and microglia (TAMs) and enhance their uptake into these cells, >10-fold higher than non-glycosylated PAMAM-OH dendrimers.

In some embodiments, the carbohydrate moieties are monosaccharides. Exemplary monosaccharides include glucose, glucosamine, galactose, mannose, fructose, dehydroascorbic acid, urate, myo-inositol. In some embodiments, the dendrimers are conjugated to one or more monosaccharides other than galactose. In preferred embodiments, hydroxyl-terminated dendrimers are modified with one or more glucose moieties to the dendrimer (“D-Glu”).

In some embodiments, the carbohydrate moieties are oligosaccharides which terminate in one or more monosaccharides including glucose, glucosamine, mannose, fructose, thus exposing these sugar moieties on the surface for binding.

i. Glucose

Glucose is a simple monosaccharide sugar with the molecular formula C₆H₁₂O₆ and a mass of 180.156 Da. The structure of glucose is set forth in Formula I. Naturally occurring form of glucose is d-glucose, while 1-glucose is produced synthetically in comparatively small amounts and is of lesser importance. The glucose molecule is an aldohexose, containing six carbon atoms and an aldehyde group, and can exist in an open-chain (acyclic) as well as ring (cyclic) form.

Glucose is naturally occurring and is found in fruits and other parts of plants in its free state. In animals, glucose is released from the breakdown of glycogen in a process known as glycogenolysis. The d-isomer, (d-glucose), also known as dextrose, occurs widely in nature, but the 1-isomer, 1-glucose, does not. Glucose can be synthesized by hydrolysis of carbohydrates such as milk sugar (lactose), cane sugar (sucrose), maltose, cellulose, glycogen, etc. Dextrose is commonly commercially manufactured from cornstarch in the US and Japan, from potato and wheat starch in Europe, and from tapioca starch in tropical areas.

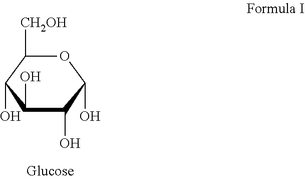

ii. Receptor-Mediated Uptake of Glycosylated Dendrimers

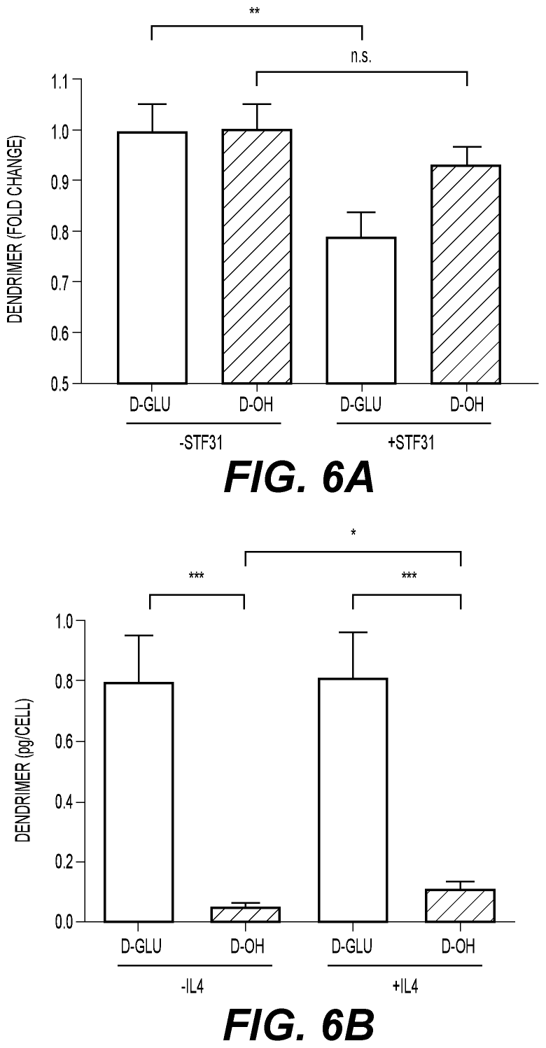

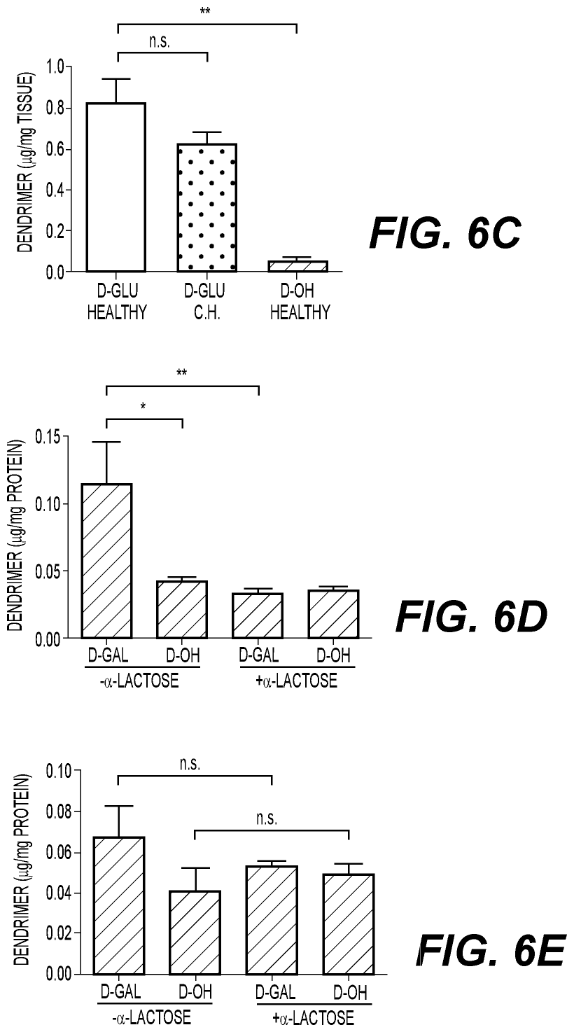

It has been demonstrated that compositions of dendrimers conjugated with glucose target and localize within brain tissue via interaction with glucose receptors on the surface of cells such as endothelial cells (Example 2 and FIG. 6A). Therefore, compositions of D-Glu facilitate uptake via glucose receptor binding in vivo.

### a. Glucose Transporters

Glucose transporters (GLUT) are a wide group of membrane proteins that facilitate the transport of glucose across the plasma membrane, a process known as facilitated diffusion. Since glucose is a vital source of energy, these transporters are present in all phyla, and the GLUT family are a family of Facilitative glucose transporter proteins that bind and transport glucose molecules into mammalian cells. GLUT is a type of uniporter transporter protein, and there are 14 different GLUTS (GLUT1-GLUT14) encoded by human genome.

GLUT proteins transport glucose and related hexoses according to a model of alternate conformation, which predicts that the transporter exposes a single substrate binding site toward either the outside or the inside of the cell. Binding of glucose to one site provokes a conformational change associated with transport, and releases glucose to the other side of the membrane. The inner and outer glucose-binding sites are, it seems, located in transmembrane segments.

Glucose uptake in microglia is facilitated predominately by GLUT1, particularly under inflammatory conditions. The excessive growth of malignant cells of a tumor requires high energy, which may come in the form of glucose, and it may be that there is an increased expression of glucose transporters in tumor cells and/or in the tumor microenvironment.

Active targeting via glucose receptors, which are expressed on endothelial cells and microglia within the brain, enables glucose-receptor specific nanocarriers to attain enhanced penetration across the blood-brain-barrier and throughout the brain tissue, while the hydroxyl groups present on the dendrimer enables specific targeting and increased drug accumulation to the activated microglia and macrophages within the brain, including TAMs within the tumor environment.

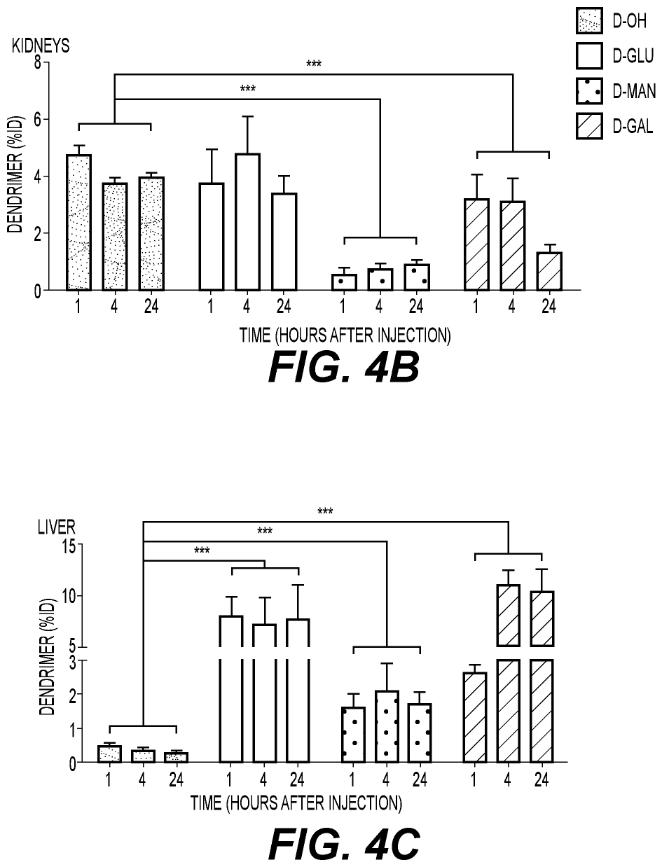

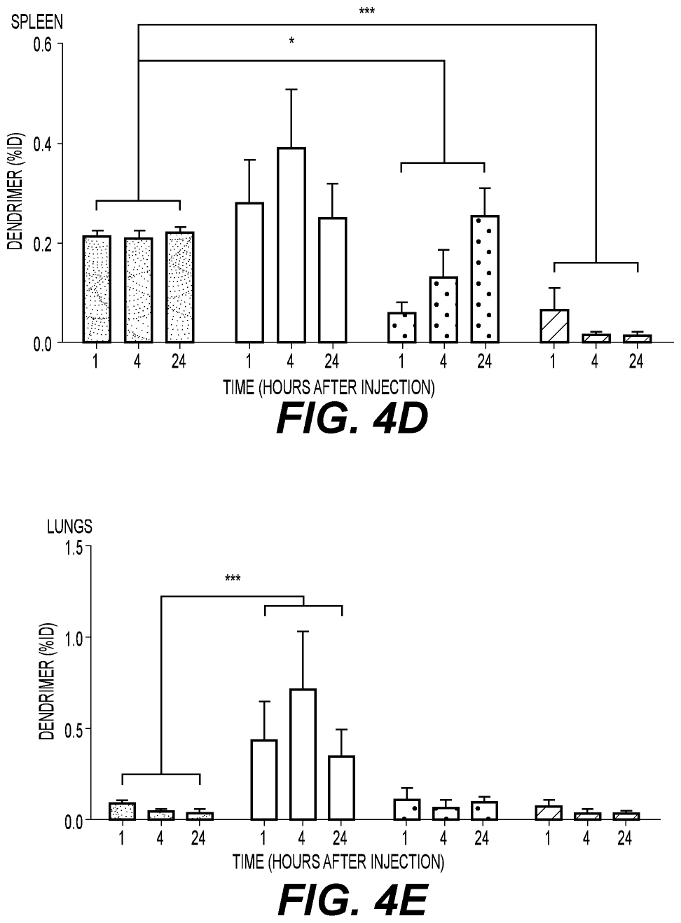

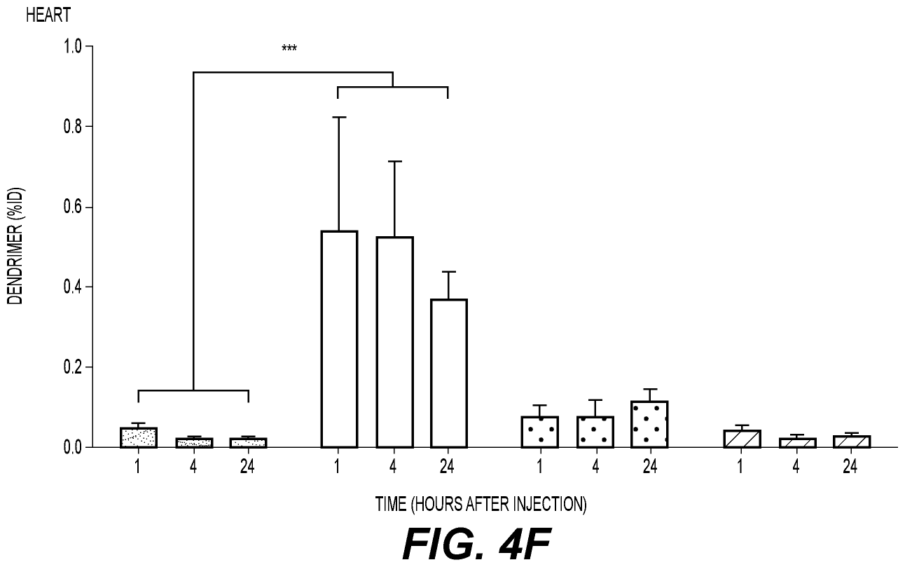

The mechanism of cellular internalization was determined to be primarily receptor-mediated transport, as evidenced by blocking the GLUT receptors with the specific inhibitor STF-31. The internalized D-Glu was found to be non-toxic to a number of common human cell lines in vitro and showed no obvious signs of toxicity to clearance organs in vivo, clearing intact from the body. Upon systemic administration in healthy mice, dendrimers rapidly cleared from the body, with less than 1% of the initial injected dose remaining per mL of plasma after 24 hours (FIGS. 4A-4E). Dendrimer internalization in resting and IL4 activated TAMs-like microglia was significantly greater with D-GLU than D-OH, indicating improved cellular internalization (FIGS. 6A-6E).

It has been shown in the Examples that the surface glucose sugars create a multivalent binding effect to GLUT, allowing the glucosylated dendrimers to effectively be transported across the blood-brain-barrier, whilst maintaining sufficient terminal-OH groups to and selectively target and internalize in microglia in vitro and in vivo. D-Glu has shown to be a highly specific delivery vehicle in selectively targeting microglia.

Accumulation of D-Glu in the brain is greatly increased relative to unmodified D-OH. D-OH exhibited a tumor/contralateral hemisphere ratio of 3.4±1.0 24 hours after administration, while D-GLU exhibited 18.8±5.4. Other sugars conjugated to dendrimers also showed increased concentrations in tumor vs contralateral hemisphere, with mannose-conjugated dendrimer (D-MAN) exhibiting 4.0±0.4, and galactose-conjugated dendrimer (D-GAL) exhibiting 7.1±1.7. Compared to quantification of liposomal nanoparticle tumor targeting in an orthotopic brain tumor model, D-GLU demonstrated ˜100-fold greater tumor accumulation, while D-MAN and D-GAL exhibited ˜8-fold greater tumor accumulation. Furthermore, there is rapid off-target clearance of D4-Glu with the plasma containing less than 1% of the original injected dose after just 24 hours.

Therefore, tissues and cells having GLUT receptors can facilitate the active uptake and intracellular transport of glycosylated dendrimers. It may be that the presence of GLUT receptors on one or more organs or tissues provides a mechanism for enhanced delivery of active agents by glucosylated dendrimers into and throughout these tissues and associated tissues. A listing of GLUT receptors and tissue distribution is set forth in Table 1, below.

TABLE 1
GLUT receptors and tissue distribution (Long, et al.,
Cell Health and Cytoskeleton; 7: 167-183 (2015)).

| GLUT | GLUT | Gene | Tissue | Substrate |
|---|---|---|---|---|
| Classes | isoforms | name | Distribution | specificity |
| I | GLUTI | SLC2AI | Red blood | Glucose/Galactose/ |
| | | | cells, | Dehydroacetic Acid |
| | | | Ubiquitous | |
| I | GLUT3 | SLC2A3 | Neurons | Glucose/Galatose/ |
| | (GLUT14) | (SLC2AI4) | (Testis) | Dehydroacetic Acid |
| I | GLUT4 | SLC2A4 | Muscle cells, | Glucose/ |
| | | | Fat cells | Dehydroacetic Acid |
| | | | (Adipocytes) | |
| I | GLUT2 | SLC2A2 | Intestine, | Glucose/Fructose/ |
| | | | Liver Kidney, | Galatose/ |
| | | | Beta cells | Glucosamine/ |
| | | | | Dehydroacetic Aci5 |
| II | GLUT5 | SLC2A5 | Intestine, | Fructose |
| | | | Kidney | |
| | | | Muscle, | |
| | | | Sperm, Brain | |
| II | GLUT7 | SLC2A7 | Intestine, | Fructose/Glucose |
| | | | Colon | |
| II | GLUT9 | SLC2A9 | Kidney, Liver, | Urate/Fructose/ |
| | | | Placenta, | Glucose |
| | | | Colon | |
| II | GLUTII | SLC2AII | Muscle, Heart | Glucose |
| | | | Placenta, | |
| | | | Kidney, | |
| | | | Pancreas, Fat | |
| III | GLUT6 | SLC2A6 | Brain, Spleen | Glucose |
| III | GLUT8 | SLC2A8 | Testes, Brain, | Glucose/Fructose |
| | | | Fat, Liver, | |
| | | | Spleen | |
| III | GLUTI0 | SLC2AI0 | Heart, Lung | Glucose |
| III | GLUTI2 | SLC2AI2 | Insulin- | Glucose/Fructose |
| | | | sensitive | |
| | | | tissues | |
| III | GLUTI3 | SLC2AI3 | Brain | Mo-inositol |
| | (HMIT) | | | |

Thus, in some embodiments, the dendrimers are conjugated to one or more carbohydrates moieties that have affinity to and are suitable for transport via one or more of GLUT1, GLUT2, GLUT3, GLUT4, GLUT5, GLUT6, GLUT7, GLUT8, GLUT9, GLUT10, GLUT11, GLUT12, GLUT13, and GLUT14. In preferred embodiments, the dendrimers are conjugated to one or more carbohydrates moieties that have a specificity towards GLUT1. In further preferred embodiments, the dendrimers are conjugated to one or more glucose and/or glucosamine moieties. In other embodiments, the dendrimers are conjugated to one or more oligosaccharides terminating in glucose and/or glucosamine moieties, i.e., glucose and/or glucosamine moieties are exposed on the surface of the dendrimer conjugates suitable for binding to one or more of the GLUTs.

iii. Exemplary Dendrimers for GLUT-Uptake

Glycosylated dendrimers including one or more active agents are provided for highly efficient and selective delivery of active agents to tumors within the brain.

In preferred embodiments, the glycosylated dendrimer is a hydroxyl-terminated dendrimer conjugated to one or more carbohydrate moieties that can be transported via GLUT transporters. The principal physiological substrate of GLUT1 is clearly glucose but it is also capable of transporting glucosamine, galactose, mannose, fructose, dehydroascorbic acid, urate, myo-inositol.

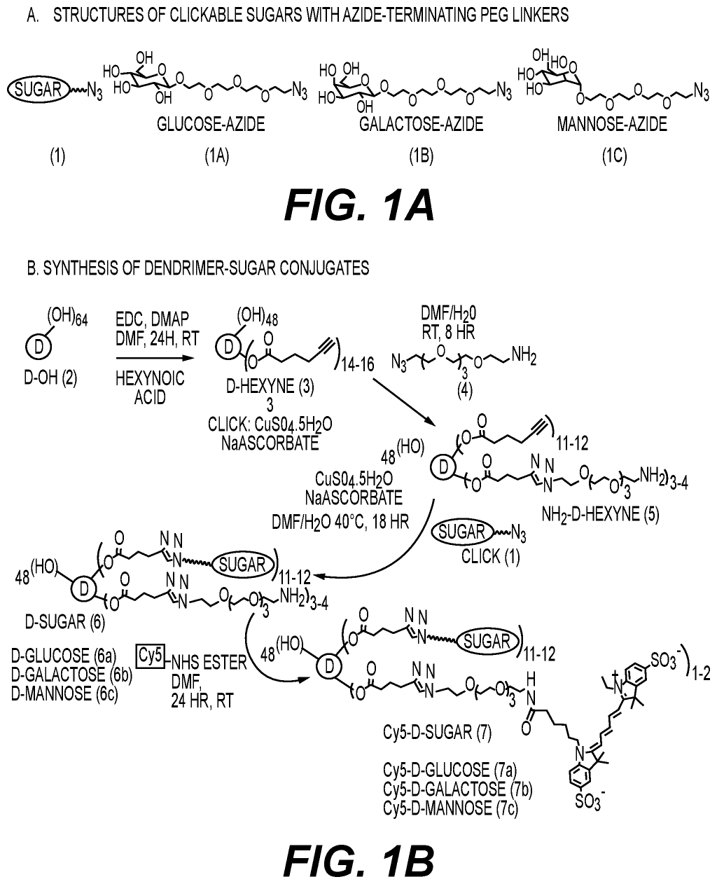

In one embodiment, the glycosylated dendrimer is a G4 (generation 4) PAMAM dendrimer including glucose and oligoethylene glycol building blocks, as shown in FIGS. 1A-1B. In some embodiments, the dendrimer (D4-Glu) includes 12-14 glucose, 48 OH groups and 2 or more active agents, such as Cy-5 dye depicted in FIG. 1B.

In one embodiment, the glycose-modified dendrimer is a generation 4 hydroxyl-terminated PAMAM dendrimer, modified with 10-12 glucose molecules (D4-Glu-10/12) on the surface, as shown in FIG. 1B, including one or more therapeutic agents for delivery to TAMs in the brain of a subject un need thereof.

Typically, carbohydrate molecules such as monosaccharides, e.g., glucose, are present in an amount by weight that is between about 1% and 40% of the total weight of the glycosylated dendrimer, for example, between about 2% and 20%, between about 5% and 15%, or between 9% and 12% of the total weight of the glycosylated dendrimer. For example, in some embodiments, the carbohydrate moieties are present in an amount that is about 1%, 2%, 3%, 4%, 5%, 6%, 7%, 8%, 9%, 10%, 11%, 12%, 13%, 14%, 15%, 16%, 17%, 18%, 19%, or 20% of the total weight of the glycosylated dendrimer following conjugation. In some embodiments, conjugation of carbohydrate molecules through one or more surface groups occurs via about 5%, 6%, 7%, 8%, 9%, 10%, 15%, 20%, or 25% of the total available surface functional groups, preferably hydroxyl groups, of the dendrimers prior to the conjugation. In other embodiments, the conjugation of carbohydrate molecules occurs on less than 5%, less than 10%, less than 15%, less than 20%, less than 25%, less than 30%, less than 35%, less than 40% of total available surface functional groups of the dendrimers prior to the conjugation. In preferred embodiments, dendrimers are conjugated to an effective amount of carbohydrate molecules for binding to GLUT-1 and/or targeting to microglia, whilst conjugated to an effective amount of active agents to treat, prevent, and/or image a tumor.

### a. Glucosamine-Conjugated Dendrimers

While glucose has a strong affinity for GLUT-1, another sugar ligand that binds to three members of the GLUT family of transporter proteins (GLUT1, 2, 4) is glucosamine (GlcN), as set forth in Formula II.

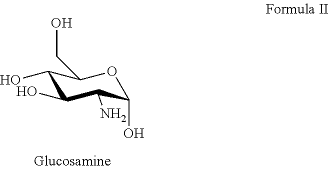

GLUT1 and GLUT4 have similar apparent affinities for glucose and GlcN, and GLUT2 has a ˜20-fold higher affinity for GlcN than for glucose. Therefore, in some embodiments, the dendrimers are modified by attachment of one or more molecules of glucosamine. For example, in some embodiments, conjugation of glucosamine molecules through one or more surface groups occurs via about 5%, 6%, 7%, 8%, 9%, 10%, 15%, 20%, or 25% of the total available surface functional groups, preferably hydroxyl groups, of the dendrimers prior to the conjugation. In other embodiments, the conjugation of glucosamine molecules occurs on less than 5%, less than 10%, less than 15%, less than 20%, less than 25%, less than 30%, less than 35%, less than 40%, less than 45%, less than 50% of total available surface functional groups of the dendrimers prior to the conjugation. In other embodiments, the dendrimers are conjugated to one or more molecules of glucose and one or more molecules of glucosamine. For example, in some embodiments, the dendrimers are conjugated to an equal number of glucose molecules and glucosamine molecules. In other embodiments, the dendrimers are conjugated to a greater number of glucose molecules than glucosamine molecules. In other embodiments, the dendrimers are conjugated to a greater number of glucosamine molecules than glucose molecules. In preferred embodiments, the total amount of glucose and glucosamine molecules conjugated to a dendrimer through one or more surface groups occupy about 5%, 6%, 7%, 8%, 9%, 10%, 15%, 20%, or 25% of the total available surface functional groups, preferably hydroxyl groups, of the dendrimer prior to the conjugation. In other embodiments, the total amount of glucose and glucosamine molecules conjugated occupy less than 5%, less than 10%, less than 15%, less than 20%, less than 25%, less than 30%, less than 35%, less than 40%, less than 45%, less than 50% of total available surface functional groups of the dendrimers prior to the conjugation.

### b. Mannose-Conjugated Dendrimers

GLUT-1 is also capable of transporting mannose. A structure of mannose is shown below in Formula III.

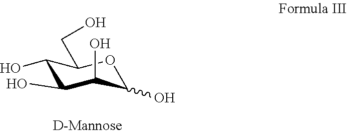

In some embodiments, the dendrimers are modified by attachment of one or more molecules of mannose. For example, in some embodiments, conjugation of mannose molecules through one or more surface groups occurs via about 5%, 6%, 7%, 8%, 9%, 10%, 15%, 20%, or 25% of the total available surface functional groups, preferably hydroxyl groups, of the dendrimers prior to the conjugation. In other embodiments, the conjugation of mannose molecules occurs on less than 5%, less than 10%, less than 15%, less than 20%, less than 25%, less than 30%, less than 35%, less than 40%, less than 45%, less than 50% of total available surface functional groups of the dendrimers prior to the conjugation. In other embodiments, the dendrimers are conjugated to one or more molecules of glucose, mannose, and glucosamine. In preferred embodiments, the total amount of monosaccharide molecules conjugated to a dendrimer through one or more surface groups occupy about 5%, 6%, 7%, 8%, 9%, 10%, 15%, 20%, or 25% of the total available surface functional groups, preferably hydroxyl groups, of the dendrimer prior to the conjugation. In other embodiments, the total amount of monosaccharide molecules conjugated occupy less than 5%, less than 10%, less than 15%, less than 20%, less than 25%, less than 30%, less than 35%, less than 40%, less than 45%, less than 50% of total available surface functional groups of the dendrimers prior to the conjugation.

### C. Coupling Agents and Spacers

Dendrimer complexes can be formed of therapeutically agents or compounds conjugated or attached to a dendrimer, a dendritic polymer or a hyperbranched polymer. Optionally, the active agents are conjugated to the dendrimers via one or more spacers/linkers via different linkages such as disulfide, ester, carbonate, carbamate, thioester, hydrazine, hydrazides, and amide linkages. The one or more spacers/linkers between a dendrimer and an agent can be designed to provide a releasable or non-releasable form of the dendrimer-active complexes in vivo. In some embodiments, the attachment occurs via an appropriate spacer that provides an ester bond between the agent and the dendrimer. In some embodiments, one or more spacers/linkers between a dendrimer and an agent are added to achieve desired and effective release kinetics in vivo.

i. Coupling Agents

In some embodiments, the active agents are attached to the dendrimer via a linking moiety that is designed to be cleaved in vivo. The linking moiety can be designed to be cleaved hydrolytically, enzymatically, or combinations thereof, to provide for the sustained release of the agents in vivo. Both the composition of the linking moiety and its point of attachment to the agent, are selected so that cleavage of the linking moiety releases either an active agent, or a suitable prodrug thereof. The composition of the linking moiety can also be selected in view of the desired release rate of the agents.

In some embodiments, the attachment occurs via one or more of disulfide, ester, ether, thioester, carbamate, carbonate, hydrazine, or amide linkages. In preferred embodiments, the attachment occurs via an appropriate spacer that provides an ester bond or an amide bond between the agent and the dendrimer depending on the desired release kinetics of the agent.

Linking moieties generally include one or more organic functional groups. Examples of suitable organic functional groups include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety is chosen in view of the desired release rate of the agents. In addition, the one or more organic functional groups can be selected to facilitate the covalent attachment of the agents to the dendrimers.

ii. Spacers

In certain embodiments, the linking moiety includes one or more of the organic functional groups described above in combination with a spacer group. The term “spacers” includes compositions used for linking a therapeutically agent to the dendrimer. The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminations.

In preferred embodiments, the attachment of the active to the dendrimer occurs via an appropriate spacer that provides a disulfide bridge between the active agent and the dendrimer. In one embodiment, the dendrimer-glucose complexes rapidly release the agent by thiol exchange reactions, under the reduced conditions found in vivo.

In some embodiments, the spacer group is composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the agents in vivo. In embodiments where the linking moiety includes a spacer group, one or more organic functional groups will generally be used to connect the spacer group to both the active agent and the dendrimers. In some embodiments, the spacer is chosen from among a class of compounds terminating in sulfhydryl, thiopyridine, succinimidyl, maleimide, vinylsulfone and carbonate group. In some embodiments, the spacer includes thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP. In some embodiments, the spacer includes peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). In some embodiments, the spacer is a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. In some embodiments, the spacer is thiosalicylic acid and its derivatives, (4-succinimidyloxycarbonyl-methyl-alpha-2-pyridylthio)toluene, (3-[2-pyridithio]propionyl hydrazide. In other embodiments, the spacer has maleimide terminations wherein the spacer includes polymer or small chemical entity such as bis-maleimido diethylene glycol and bis-maleimido triethylene glycol, bis-maleimidoethane, bismaleimidohexane. In some embodiments, the spacer includes vinylsulfone such as 1,6-Hexane-bis-vinylsulfone. In some embodiments, the spacer is a thioglycoside such as thioglucose. In some embodiments, the spacer is a reduced protein such as bovine serum albumin and human serum albumin, any thiol terminated compound capable of forming disulfide bonds. In some embodiments, the spacer includes polyethylene glycol having maleimide, succinimidyl and thiol terminations.

### D. Active Agents

The glycosylated dendrimers complexed to, covalently conjugated to, or having intra-molecularly dispersed or encapsulated therein one or more active agents are described.

The glycosylated dendrimer conjugates include one or more active agents for delivery selectively to TAMs in vivo. Agents to be included in the glycosylated dendrimer conjugates to be delivered to target cells can be proteins or peptides, sugars or carbohydrate, nucleic acids or oligonucleotides, lipids, small molecules (e.g., molecular weight less than 2,000 Dalton, preferably less than 1,500 Dalton, more preferably 300-700 Dalton), or combinations thereof. The nucleic acid can be an oligonucleotide encoding a protein, for example, a DNA expression cassette or an mRNA. Representative oligonucleotides include siRNAs, microRNAs, DNA, and RNA. In some embodiments, the agent is a therapeutic antibody.

Dendrimers have the advantage that multiple therapeutic, prophylactic, and/or diagnostic agents can be delivered with the same dendrimers. One or more types of agents can be encapsulated, complexed, or conjugated to the dendrimer. In one embodiment, the dendrimers are complexed with or conjugated to two or more different classes of agents, providing simultaneous delivery with different or independent release kinetics at the target site. In another embodiment, the dendrimers are covalently linked to at least one detectable moiety and at least one class of agents. In a further embodiment, dendrimer complexes each carrying different classes of agents are administered simultaneously for a combination treatment.

Active agents can include those that alleviate or treat one or more symptoms of one or more brain cancers. Exemplary active agents are immune-modulatory agents and chemotherapeutic agents.

i. Therapeutic and Prophylactic Agents

The glycosylated dendrimer conjugates preferably include one or more therapeutic, prophylactic, or prognostic agents that are complexed or conjugated to the dendrimers. Representative therapeutic agents include, but are not limited to, anti-cancer agents, immune-modulatory agents, antioxidants, anti-infectious agents, and combinations thereof.

### a. Anti-Cancer Chemotherapeutic Agents

In some embodiments, the compositions include one or more anti-cancer agents. Therefore, in some embodiments, glycosylated dendrimer conjugates preferably include one or more chemotherapeutic agents. The majority of chemotherapeutic drugs can be divided into alkylating agents, antimetabolites, anthracyclines, plant alkaloids, topoisomerase inhibitors, and other anti-tumor agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

In some embodiments, the active agents are histone deacetylase (HDAC) inhibitors. In one embodiment, the active agent is vorinostat. In other embodiments, the active agents are topoisomerase I and/or II inhibitors. In a particular embodiment, the active agent is etoposide or camptothecin.

Additional anti-cancer agents include, but are not limited to, irinotecan, exemestane, octreotide, carmofur, clarithromycin, zinostatin, tamoxifen, tegafur, toremifene, doxifluridine, nimustine, vindensine, nedaplatin, pirarubicin, flutamide, fadrozole, prednisone, medroxyprogesterone, mitotane, mycophenolate mofetil, and mizoribine.

Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYE001) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sirna Therapeutics); multifunctional antiangiogenic agents such as NEOVASTAT® (AE-941) (Aeterna Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

Representative chemotherapeutics commonly used in treating brain tumors include taxols such as paclitaxel, BCNU, camptothecin, doxycycline, cisplatin, and derivatives, analogues and prodrugs thereof. Examples of PD-1 inhibitors include, for example, MDX-1106 is a genetically engineered, fully human immunoglobulin G4 (IgG4) monoclonal antibody specific for human PD-1, and pembrolizumab, recently approved by the US FDA.

### b. Immunomodulatory Agents

In some embodiments the compositions include one or more immune-modulating drugs. Exemplary immune-modulating drugs include cyclosporine, tacrolimus and rapamycin.

The dendrimer complexes include one or more therapeutic agents that are immunomodulatory agents. The term “immunomodulatory agent” and “immunotherapeutic agent” mean an active agent that elicits a specific effect upon the immune system of the recipient. Immunomodulation can include suppression, reduction, enhancement, prolonging or stimulation of one or more physiological processes of the innate or adaptive immune response to antigen, as compared to a control. Typically, immunomodulatory agents can modulate immune microenvironment for a desired immunological response (e.g., increasing anti-tumor activity) by targeting one or more immune cells or cell types at a target site, and thus, are not necessarily specific to any cancer type. In some embodiments, the immunomodulatory agents are specifically delivered to kill, inhibit, or reduce activity or quantity of suppressive immune cells such as tumor-associated macrophages for an enhanced anti-tumor response at a tumor site.

In some embodiments, dendrimers associated with or conjugated to one or more immunomodulatory agents are used in combination with anti-tumor vaccines and/or adoptive cell therapy (ACT) as an adjuvant, for example to increase expression of innate immune genes, infiltration and expansion of activated effector T cells, antigen spreading, and durable immune responses.

In some embodiments, the immunomodulatory agents are any inhibitors targeting one or more of EGFR, ERBB2, VEGFRs, Kit, PDGFRs, ABL, SRC, mTOR, and combinations thereof. In some embodiments, the immunomodulatory agents are one or more inhibitors and analogues thereof, such as crizotinib, ceritinib, alectinib, brigatinib, bosutinib, dasatinib, imatinib, nilotinib, ponatinib, vemurafenib, dabrafenib, ibrutinib, palbociclib, sorafenib, ribociclib, cabozantinib, gefitinib, erlotinib, lapatinib, vandetanib, afatinib, osimertinib, ruxolitinib, tofacitinib, trametinib, axitinib, lenvatinib, nintedanib, pazopanib, regorafenib, sorafenib, sunitinib, vandetanib, bosutinib, dasatinib, dacomitinib, ponatinib, and combinations thereof. In some embodiments, the immunomodulatory agents are tyrosine kinase inhibitors such as HER2 inhibitors, EGFR tyrosine kinase inhibitors. Exemplary EGFR tyrosine kinase inhibitors include gefitinib, erlotinib, afatinib, dacomitinib, and osimertinib.

Additional immunomodulatory agents can include one or more cytotoxic agents that are toxic to one or more immune cells, thus can kill/inhibit one or more types of suppressive immune cells. When delivered selectively to target immune cells such as being conjugated to dendrimers, these agents are able to selectively kill suppressive immune cells and thus alter immunological microenvironment in and around tumors.

ii. Diagnostic Agents

In some cases, the agents delivered to the target cells or tissues via glycosylated dendrimer are diagnostic agents. Examples of diagnostic agents that can be delivered to the brain by glycosylated dendrimer conjugates include paramagnetic molecules, fluorescent compounds, magnetic molecules, and radionuclides, x-ray imaging agents, and contrast media. Examples of other suitable contrast agents include gases or gas emitting compounds, which are radioopaque. Glycosylated dendrimer conjugates can further include agents useful for determining the location of administered compositions. Agents useful for this purpose include fluorescent tags, radionuclides and contrast agents.

Exemplary diagnostic agents include dyes, fluorescent dyes, near infra-red dyes, SPECT imaging agents, PET imaging agents and radioisotopes. Representative dyes include carbocyanine, indocarbocyanine, oxacarbocyanine, thiicarbocyanine and merocyanine, polymethine, coumarine, rhodamine, xanthene, fluorescein, boron-dipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Exemplary SPECT or PET imaging agents include chelators such as di-ethylene tri-amine penta-acetic acid (DTPA), 1,4,7,10-tetra-azacyclododecane-1,4,7,10-tetraacetic acid (DOTA), di-amine dithiols, activated mercaptoacetyl-glycyl-glycyl-gylcine (MAG3), and hydrazidonicotinamide (HYNIC).

Exemplary isotopes include Tc-94m, Tc-99m, In-111, Ga-67, Ga-68, Gd3+, Y-86, Y-90, Lu-177, Re-186, Re-188, Cu-64, Cu-67, Co-55, Co-57, F-18, Sc-47, Ac-225, Bi-213, Bi-212, Pb-212, Sm-153, Ho-166, and Dy-166.

In preferred embodiments, the dendrimer compositions include one or more radioisotopes suitable for positron emission tomography (PET) imaging. Exemplary positron-emitting radioisotopes include carbon-11 (¹¹C), copper-64 (⁶⁴Cu), nitrogen-13 (¹³N), oxygen-15 (¹⁵O), gallium-68 (⁶⁸Ga), and fluorine-18 (¹⁸F), e.g., 2-deoxy-2-¹⁸F-fluoro-β-D-glucose (¹⁸F-FDG).

In further embodiments, a singular glycosylated dendrimer conjugate composition can simultaneously treat and/or diagnose a disease or a condition at one or more locations in the body.

### E. Additional Targeting or Binding Moieties

Glycosylated dendrimers having terminal-OH groups target to activated microglia within the brain, including tumor-associated microglia cells (TAMs). In some embodiments, the glycosylated dendrimer includes one or more additional tissue targeting or tissue binding moieties, for targeting the dendrimer to one or more additional specific location in vivo, and/or for enhancing the in vivo residence time at a desired location within the body. For example, in some embodiments, the glycosylated dendrimer is sequestered or bound to one or more distinct tissues or organs following local or systemic administration into the body. Therefore, the presence of a targeting or binding moiety can enhance the delivery of an active agent to a target site relative to the glycosylated dendrimer and active agent in the absence of a targeting or binding moiety. Conjugation of the glycosylated dendrimer to one or more targeting or binding moieties can be via a spacer, and the linkage between the spacer and dendrimer, and/or the spacer and targeting agent can be designed to provide releasable or non-releasable forms of the glycosylated dendrimer-targeting agent complex. In some embodiments, the targeting agent also has a therapeutic effect at the targeted site.

### F. Glycosylated Dendrimer Complexes

The hydroxyl surface groups allow for the attachment of small molecules, imaging agents, and small biological agents such as siRNA regardless of the payload's charge or aqueous solubility. Dendrimers modified with glucose and/or glucosamine can include one or more therapeutic or prophylactic agents complexed, covalently conjugated, or intra-molecularly dispersed or encapsulated with the dendrimer. Conjugation of one or more agents to the dendrimer component of a dendrimer-Glu complex can occur prior to, at the same time as, or subsequent to conjugation of the dendrimer with the glucose. Compositions and methods for conjugating agents with dendrimers are known in the art and for example, as described in U.S. Published Application Nos. US 2011/0034422, US 2012/0003155, and US 2013/0136697.

In some embodiments, one or more agents are covalently attached to the dendrimer component of the glycosylated dendrimer. In some embodiments, glycosylated dendrimer conjugates include one or more active agents conjugated or complexed with the glycosylated dendrimer via one or more linking moieties. In further embodiments, the linking moieties incorporate or are conjugated with one or more spacer moieties. The linking and/or spacer moieties can be cleavable, for example, by exposure to the intracellular compartments of target cells in vivo. The active agent and/or targeting moiety can be either covalently attached or intra-molecularly dispersed or encapsulated. The glycosylated dendrimer is preferably a PAMAM dendrimer from generation 0, up to generation 10, having hydroxyl terminations. In preferred embodiments, the glycosylated dendrimer is linked to agents via a spacer ending in disulfide, ester, or amide bonds.

Reactions and strategies useful for the covalent attachment of agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent attachment of a given agent can be selected in view of the linking moiety desired, as well as the structure of the agents and dendrimers as a whole as it relates to compatibility of functional groups, protecting group strategies, and the presence of labile bonds.

The optimal drug loading will necessarily depend on many factors, including the choice of drug, dendrimer structure and size, and tissues to be treated. In some embodiments, the one or more active agents are encapsulated, associated, and/or conjugated to the dendrimer component of the dendrimer-glucose complex at a concentration of about 0.01% to about 45%, preferably about 0.1% to about 30%, about 0.1% to about 20%, about 0.1% to about 10%, about 1% to about 10%, about 1% to about 5%, about 3% to about 20% by weight, and about 3% to about 10% by weight. However, optimal drug loading for any given drug, dendrimer, and site of target can be identified by routine methods, such as those described.

In some embodiments, conjugation of a dendrimer to an active agent occurs prior to conjugation of the dendrimer with glucose. In some embodiments, conjugation of active agents and/or linkers to dendrimer-glucose occurs through one or more surface and/or interior groups. Thus, in some embodiments, the conjugation of agents/linkers occurs via about 1%, 2%, 3%, 4%, or 5% of the total available surface functional groups, preferably hydroxyl groups, of the dendrimers prior to the conjugation. In other embodiments, the conjugation of agents/linkers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation and/or the modification with glucose and/or active agents. In preferred embodiments, dendrimer complexes retain an effective amount of surface functional groups for modification with glucose for targeting to target cells, whilst conjugated to an effective amount of agents for treat, prevent, and/or image the disease or disorder.

Generally, one or more active agents are encapsulated, associated, and/or conjugated in the glycosylated dendrimer complex at an amount of about 0.01% to about 30%, preferably about 1% to about 20%, more preferably about 5% to about 20% by weight.

Typically, dendrimer complexes have a hydrodynamic volume in the nanometer range. For example, in some embodiments, the glycosylated dendrimer complex including one or more active agents complexed with or conjugated to the dendrimer has a diameter of about 2 nm to about 100 nm, or more than 100 nm, up to 500 nm, depending upon the generation of dendrimer, the chemical composition and amount of active agent loaded. Preferably, a glycosylated dendrimer complex including one or more active agents complexed with or conjugated to the dendrimer has a size that is optimal for passage into and throughout the brain tissue. In some embodiments, the glycosylated dendrimer complex including one or more active agents complexed with or conjugated to the dendrimer has a near neutral zeta potential. The term “neutral surface charge” of a glycosylated dendrimer complex refers to an electrokinetic potential (zeta-potential) of the complex that is 0 mV. Therefore, in some embodiments, a glycosylated dendrimer complex has a zeta-potential that is approximately 0 mV, such as from −10 mV to 10 mV, from −5 mV to 5 mV, preferably from −1 mV to 1 mV.

### III. Pharmaceutical Formulations

Pharmaceutical compositions including glycosylated dendrimers and one or more active agents may be formulated in a conventional manner using one or more physiologically acceptable carriers including excipients and auxiliaries which facilitate processing of the active compounds into preparations which can be used pharmaceutically.

Proper formulation is dependent upon the route of administration chosen. In preferred embodiments, the compositions are formulated for parenteral delivery. In some embodiments, the compositions are formulated for intravenous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain one or more galactosylated dendrimer complexes in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS) and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

The compositions of glycosylated dendrimer are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine effective doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD₅₀ (the dose is lethal to 50% of the population). The dose ratio of toxic to therapeutic effects is the therapeutic index, and is expressed as the ratio, LD₅₀/ED₅₀. Pharmaceutical compositions which exhibit large therapeutic indices are preferred. The data obtained from cell culture assays and animal studies can be used in formulating a range of dosages for human use.

In certain embodiments, the compositions of glycosylated dendrimer are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions of glycosylated dendrimer formulated for administration by parenteral (intramuscular, intraperitoneal, intravenous or subcutaneous injection) and enteral routes of administration are described.

### A. Parenteral Administration

The compositions of glycosylated dendrimer can be administered parenterally. The phrases “parenteral administration” and “administered parenterally” are art-recognized terms, and include modes of administration other than enteral and topical administration. The dendrimers can be administered parenterally, for example, by subdural, intravenous, intrathecal, intraventricular, intraarterial, intra-amniotic, intraperitoneal, or subcutaneous routes.

For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### B. Enteral Administration

The compositions of glycosylated dendrimer can be administered enterally. The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions, or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides and other components of infant formulas.

In preferred embodiments, the compositions are formulated for oral administration. Oral formulations may be in the form of chewing gum, gel strips, tablets, capsules or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

### IV. Methods of Use

Carbohydrate-modified dendrimer compositions selectively bind to GLUT receptors on specific cells, including GLUT-1 receptors on microglia. The efficient binding to the GLUT-1 receptors directs selective internalization of the dendrimer-glucose across endothelial barriers and into tumor-associated microglial cells (TAMs). Methods of using glycosylated dendrimer (D-Gly) compositions are described. In some embodiments, treating using the compositions of glycosylated dendrimer reduces or inhibits the number or activity of anti-inflammatory activities of one or more cell types in a disease or disorder associated with excessive immunosuppressive environment such as in cancer cells/tissues.

Methods of inducing or increasing the expansion and/or function of pro-inflammatory and tumoricidal classically activated or M1 macrophages are also described.

### Tumor Associated Macrophages (TAMs)

Methods for modulating immune microenvironment for a desirable immunological outcome are described. In some embodiments, glycosylated dendrimer conjugates are administered to a subject to deplete, inhibit, or reduce tumor associated macrophages (TAMs, or M2-like macrophages) in the subject, for example, via blocking proliferation, migration, or activation of TAMs. Methods of selective delivery of active agents to TAMs are also provided.

The methods employ glycosylated dendrimer compositions to selectively deliver one or more active agents to TAMs in vivo with high specificity and efficacy. Therefore, in some embodiments, the methods manipulate TAMs to modulate the immune microenvironment for a desirable immunological outcome.

Methods of using glycosylated dendrimers for enhancing tumor immunogenicity and/or inducing an anti-tumor immune response are described. The methods selectively deliver, one or more active agents to TAMs for the treatment, prevention, and diagnosis of tumors, such as brain tumors.

In some embodiments, treatment using the compositions of glycosylated dendrimers reduces or inhibits the number or activity of tumor-permissive and immunosuppressive immune cells, for example, TAMs, relative to the number or activity of the tumor-permissive and immunosuppressive immune cells prior to administration of the glycosylated dendrimers, or compared to administration of the active agent absent a dendrimer scaffold, or using a dendrimer in the absence of glycosylation.

The methods include administering to the subject the glycosylated dendrimer conjugates including one or more active agents in an effective amount to deplete, inhibit or reduce TAMs. In some embodiments, the compositions of glycosylated dendrimers are administered in an amount effective to inhibit or reduce the immune suppressive functions of TAM, for example, by decreasing one or more immune suppressive or anti-inflammatory cytokines such as IL-4, IL-10, and IL-13, increasing one or more immune stimulatory cytokines such as IL-12, IL-6, IL-1b, CXCL9, CXCL10, TNFα, or combinations thereof.

Methods of treating cancer mediated or regulated by TAMs are also described. The methods include administering to the subject the glycosylated dendrimer conjugates, including one or more active agents in an effective amount to treat and/or alleviate one or more symptoms associated with cancer.

Methods of using glycosylated dendrimer conjugates for treating or preventing diseases or disorders can include the step of identifying and/or selecting a subject in need thereof. In some embodiments, the glycosylated dendrimer conjugates include an agent that is attached or conjugated to dendrimers, which is capable of preferentially releasing the drug intracellularly under the reduced conditions found in vivo. The agent can be either covalently attached or intra-molecularly dispersed or encapsulated. Typically, the amount of glycosylated dendrimer conjugates administered to the subject is selected to deliver an effective amount to reduce, prevent, or otherwise alleviate one or more clinical or molecular symptoms of the disease or disorder to be treated compared to a control, for example, a subject treated with the active agent without dendrimer, or treated with un-glycosylated dendrimer. In a preferred embodiment, the methods treat or prevent cancer in a subject in need thereof.

### A. Methods of Treating Cancer

In some embodiments, compositions of glycosylated dendrimers conjugated or complexed with one or more immunomodulatory agents and/or additional therapeutic or diagnostic agents are administered to a subject having a proliferative disease, such as a benign or malignant tumor. In some embodiments, the subjects to be treated have been diagnosed with stage I, stage II, stage III, or stage IV cancer.

The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods are useful for treating subjects having benign or malignant tumors by delaying or inhibiting the growth of a tumor in a subject, reducing the growth or size of the tumor, inhibiting, or reducing metastasis of the tumor, and/or inhibiting or reducing symptoms associated with tumor development or growth.

Malignant tumors which may be treated are classified according to the embryonic origin of the tissue from which the tumor is derived. Carcinomas are tumors arising from endodermal or ectodermal tissues such as skin or the epithelial lining of internal organs and glands. The compositions are particularly effective in treating carcinomas. Sarcomas, which arise less frequently, are derived from mesodermal connective tissues such as bone, fat, and cartilage. The leukemias and lymphomas are malignant tumors of hematopoietic ceils of the bone marrow. Leukemias proliferate as single cells, whereas lymphomas tend to grow as tumor masses. Malignant tumors may show up at numerous organs or tissues of the body to establish a cancer.

In some embodiments, the subject to be treated is one with one or more solid tumors. A solid tumor is an abnormal mass of tissue that usually does not contain cysts or liquid areas. Solid tumors may be benign (not cancer), or malignant (cancer). Examples of solid tumors are sarcomas, carcinomas, and lymphomas. In preferred embodiments, the compositions and methods are effective in treating one or more symptoms of cancers of the brain, skin, lung, liver, pancreas, kidney, breast, prostate, colon and rectum, bladder, etc. In a further embodiment, the tumor is a focal lymphoma or a follicular lymphoma. Cancer forms that can be prevented, treated or otherwise diminished by the compositions include glioblastomas, myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, papillary adenocarcinomas, and gastric cancer (for a review see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In some embodiments, the cancers are characterized as being triple negative breast cancer, or having one or more KRAS-mutations, EGFR mutations, ALK mutations, RB1 mutations, HIF mutations, KEAP mutations, NRF mutations, or other metabolic-related mutations, or combinations thereof.

The methods and compositions as described are useful for both prophylactic and therapeutic treatment. Therapeutic treatment involves administering to a subject a therapeutically effective amount of the compositions or pharmaceutically acceptable salts thereof as described after cancer is diagnosed. In further embodiments, the compositions are used for prophylactic use i.e., prevention, delay in onset, diminution, eradication, or delay in exacerbation of signs or symptoms after onset, and prevention of relapse. For prophylactic use, a therapeutically effective amount of the compounds and compositions or pharmaceutically acceptable salts thereof as described are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms. Prophylactic administration can be used, for example, in the chemopreventative treatment of subjects presenting precancerous lesions, those diagnosed with early-stage malignancies, and for subgroups with susceptibilities (e.g., family, racial, and/or occupational) to particular cancers. The compositions can also be used to treat metastases or tumors at multiple locations.

i. Cancer

Methods of using dendrimer-glucose compositions for treating or removing one or more cancers in a subject are described. As used herein, the term “proliferative disease” includes cancer and other diseases such as benign and malignant neoplasias and hyperplasias. In some embodiments, the dendrimer compositions and formulations thereof are used in a method for treating a cancer in a subject in need of. The method for treating a cancer in a subject in need of including administering to the subject a therapeutically effective amount of the dendrimer compositions.

A cancer in a patient refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells. A tumor refers to all neoplastic cell growth and proliferation, whether malignant or benign, and all precancerous and cancerous cells and tissues. A solid tumor is an abnormal mass of tissue that generally does not contain cysts or liquid areas. A solid tumor may be in the brain, colon, breasts, prostate, liver, kidneys, lungs, esophagus, head and neck, ovaries, cervix, stomach, colon, rectum, bladder, uterus, testes, and pancreas, as non-limiting examples. In some embodiments, the solid tumor regresses or its growth is slowed or arrested after the solid tumor is treated with the presently disclosed methods. In other embodiments, the solid tumor is malignant. In some embodiments, the cancer comprises Stage 0 cancer. In some embodiments, the cancer comprises Stage I cancer. In some embodiments, the cancer comprises Stage II cancer. In some embodiments, the cancer comprises Stage III cancer. In some embodiments, the cancer comprises Stage IV cancer. In some embodiments, the cancer is refractory and/or metastatic. For example, the cancer may be refractory to treatment with radiotherapy, chemotherapy or monotreatment with immunotherapy.

Methods of using dendrimer-glucose compositions for treating one or more other cancers in a subject are described. In some embodiments, the cancer is in a tissue or organ having one or more of the GLUT1-13 receptors on the surface of the tissue or organ, or on an associated or proximal organ. In other embodiments, the cancer has metastasized from another tissue or cell type having GLUT receptors. In some embodiments, the glycosylated dendrimers selectively target and accumulate within tumor associated macrophages within and surrounding the tumor. The glycosylated dendrimers deliver active agents to the tumor associated macrophages without toxicity to surrounding non-target and/or non-cancer cells.

In some embodiments, the cancer includes newly diagnosed or recurrent cancers, including without limitation, acute lymphoblastic leukemia, acute myelogenous leukemia, advanced soft tissue sarcoma, metastatic or aggressive breast cancer, breast carcinoma, bronchogenic carcinoma, choriocarcinoma, chronic myelocytic leukemia, colon carcinoma, colorectal carcinoma, Ewing's sarcoma, gastrointestinal tract carcinoma, hepatocellular carcinoma, Hodgkin's disease, intracranial ependymoblastoma, large bowel cancer, leukemia, liver cancer, lung carcinoma, Lewis lung carcinoma, lymphoma, malignant fibrous histiocytoma, a mammary tumor, melanoma, mesothelioma, neuroblastoma, osteosarcoma, ovarian cancer, pancreatic cancer, a pontine tumor, premenopausal breast cancer, prostate cancer, rhabdomyosarcoma, reticulum cell sarcoma, sarcoma, small cell lung cancer, a solid tumor, stomach cancer, testicular cancer, and uterine carcinoma. In some embodiments, the cancer is acute leukemia. In some embodiments, the cancer is acute lymphoblastic leukemia. In some embodiments, the cancer is acute myelogenous leukemia. In some embodiments, the cancer is advanced soft tissue sarcoma. In some embodiments, the cancer is a brain cancer. In some embodiments, the cancer is breast cancer (e.g., metastatic or aggressive breast cancer). In some embodiments, the cancer is breast carcinoma. In some embodiments, the cancer is bronchogenic carcinoma. In some embodiments, the cancer is choriocarcinoma. In some embodiments, the cancer is chronic myelocytic leukemia. In some embodiments, the cancer is a colon carcinoma (e.g., adenocarcinoma). In some embodiments, the cancer is colorectal cancer (e.g., colorectal carcinoma). In some embodiments, the cancer is Ewing's sarcoma. In some embodiments, the cancer is gastrointestinal tract carcinoma. In some embodiments, the cancer is large bowel cancer. In some embodiments, the cancer is leukemia. In some embodiments, the cancer is liver cancer. In some embodiments, the cancer is lung cancer (e.g., lung carcinoma). In some embodiments, the cancer is Lewis lung carcinoma. In some embodiments, the cancer is lymphoma. In some embodiments, the cancer is malignant fibrous histiocytoma. In some embodiments, the cancer comprises a mammary tumor. In some embodiments, the cancer is melanoma. In some embodiments, the cancer is mesothelioma. In some embodiments, the cancer is neuroblastoma. In some embodiments, the cancer is osteosarcoma. In some embodiments, the cancer is ovarian cancer. In some embodiments, the cancer is pancreatic cancer. In some embodiments, the cancer comprises a pontine tumor. In some embodiments, the cancer is premenopausal breast cancer. In some embodiments, the cancer is prostate cancer. In some embodiments, the cancer is rhabdomyosarcoma. In some embodiments, the cancer is reticulum cell sarcoma. In some embodiments, the cancer is sarcoma. In some embodiments, the cancer is small cell lung cancer. In some embodiments, the cancer comprises a solid tumor. In some embodiments, the cancer is stomach cancer. In some embodiments, the cancer is testicular cancer. In some embodiments, the cancer is uterine carcinoma.

In a preferred embodiment, the methods treat or prevent brain cancer in a subject in need thereof.

### a. Brain Cancers

Methods of using dendrimer-glucose compositions for treating or removing one or more brain cancers in a subject are described. The term brain cancer, includes cancers of the CNS and brain, including, but not limited to, gliomas, glioblastoma (glioblastoma multiforme), gliosarcoma, astrocytoma, oligodendroglioma, ependymoma (intracranial ependymoblastoma), meningioma, medulloblastoma, ganglioma, head and neck squamous cell carcinoma Schwannoma, craniopharyngioma, cordomas and pituitary tumors. Therefore, in some embodiments, the cancer is a glioma. In some embodiments, the cancer is glioblastoma multiforme. In some embodiments, the cancer is head and neck squamous cell carcinoma. In some embodiments, the cancer is craniopharyngioma. In some embodiments, the cancer is gliosarcoma. In some embodiments, the cancer is intracranial ependymoblastoma.

The tumors may also be of a different origin than the brain. For example, the tumors may have originated as alveolar rhabdomyosarcoma, bone cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, colon cancer, esophageal cancer, cervical cancer, gastrointestinal carcinoid tumor. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

The compositions and methods are also suitable for prophylactic use. In some embodiments, the methods treat or prevent one or more symptoms of one or more brain cancers and/or diseases include administering to the subject glycosylated dendrimers complexed, covalently conjugated, or intra-molecularly dispersed or encapsulated with one or more therapeutic or prophylactic agents, in an amount effective to treat, alleviate or prevent one or more symptoms of one or more brain cancers and/or proliferative diseases of the brain. Typically, the methods employ glycosylated dendrimer conjugates for delivery of active agents selectively to cells and tissues associated with a brain tumor in the brain of a subject. In some embodiments, the methods selectively deliver immunomodulatory agents to TAMs within the tumor or tumor microenvironment. In other embodiments, the methods selectively deliver chemotherapeutic agents to TAMs within the tumor or tumor microenvironment. In other embodiments, the methods deliver one or more additional active agents to TAMs within the tumor or tumor microenvironment, or combinations of chemotherapeutic agents, immunomodulatory agents, and/or additional therapeutic agents. The glycosylated dendrimer conjugates may also include a targeting agent, but as demonstrated by the examples, these are not required for delivery to TAMs.

The methods of treatment can also include the step of identifying and selecting a subject in need of treatment for brain cancer and who would benefit from administration with the D-Gal compositions. Therefore, methods for treating or preventing brain cancers can include the step of identifying and/or selecting a subject in need thereof, for example, a subject having one or more symptoms of a brain cancer. Physical symptoms and clinical manifestations of brain cancer include headaches, seizures (fits), persistently feeling sick (nausea), being sick (vomiting) and drowsiness, mental or behavioral changes, such as memory problems or changes in personality, progressive weakness or paralysis on one side of the body, and vision or speech problems.

In some embodiments, the subject has been medically diagnosed as having a brain cancer by exhibiting clinical (e.g., physical) symptoms of the disease. In other embodiments, the subject has been medically diagnosed as having a brain cancer by exhibiting clinical (e.g., physical) symptoms, which are indicative of an increased risk or likelihood of developing brain cancer. Therefore, in some embodiments, formulations of the disclosed D-Gal compositions are administered to a subject prior to a clinical diagnosis of a brain cancer.

In some embodiments, the D-Gal compositions are administered in an amount effective to inhibit or reduce serum levels of one or more biomarkers associated with brain cancer. Biomarkers of brain cancer include MGMT promoter methylation as a prognostic and predictive marker in glioblastoma, co-deletion of 1p and 19q differentiating oligodendrogliomas from astrocytomas, and IDH1/2 mutations.

### b. Immune Modulation

In some embodiments, the glycosylated dendrimer conjugates are administered in combination with one or more immunomodulatory agents to a subject having a brain cancer to modulate a tumorigenic microenvironment within the region of a brain tumor in the subject.

The initiation and development of gliomas typically occurs within a strong immunosuppressive tumor microenvironment (TME) within the immune-privileged environment of the brain. The strong immunosuppressive TME of gliomas has led them to be referred to in the literature as “cold tumors”. Many studies have demonstrated that cytokines, chemokines, and regulatory immune-suppressive cells, such as TGF-β, IL-10, prostaglandin E2, NKT cells, T/B regulatory cells (T/Breg), tumor-associated macrophage/microglia (TAMs), and myeloid-derived suppressor cells (MDSCs), create a specific immunosuppressive TME, which is important for anti-tumor responses and glioma progression. Therefore, methods of treating a brain tumor in a subject include reducing, eliminating, or otherwise disrupting the immunosuppressive TME surrounding brain cancer cells in the subject. Glycosylated dendrimer compositions, including one or more active agents to treat or prevent a brain cancer can be administered to a subject to selectively deliver the active agents to the TAMs and other immune suppressive cells within the TME of brain cancers to reduce, eliminate, or otherwise disrupt the immunosuppressive factors associated with these cells, and/or induce an immunostimulatory response in the TME within the brain of the subject.

Therefore, in some embodiments, methods for treating or preventing one or more brain cancer and/or proliferative disorder of the brain include administering to the subject compositions including glucose-modified hydroxyl terminated dendrimers of generation 4, generation 5, generation 6, generation 7, or generation 8 covalently conjugated to one or more immunomodulatory agents, in an amount effective to reduce, eliminate, or otherwise disrupt the immunosuppressive TME surrounding brain cancer cells in the subject. In some embodiments, the methods administer the immunomodulatory agents, in an amount effective treat or prevent one or more symptoms brain cancer and/or proliferative disorder of the brain.

In some embodiments, the immunomodulatory agents are administered in combination with vaccine against the cancer, which may be protein or nucleic acid vaccines, such as mRNA vaccines.

### c. Chemotherapy

In some embodiments, the glycosylated dendrimer conjugates including one or more anti-cancer agents are administered to a subject having a brain cancer to reduce the number or viability of cancer cells within the region of a brain tumor in the subject. Exemplary anti-cancer agents include conventional chemotherapy drugs.

In some embodiments, methods for treating or preventing one or more brain cancer and/or proliferative disorder of the brain include administering to the subject compositions including glucose-modified hydroxyl terminated dendrimers of generation 4, generation 5, generation 6, generation 7, or generation 8 covalently conjugated to one or more anti-cancer agents, in an amount effective to treat or prevent one or more symptoms of one or more brain cancer and/or proliferative disorder of the brain.

In some embodiments, the glycosylated dendrimer compositions including one or more anti-cancer agents and/or one or more immunomodulatory agents, or formulations thereof are administered in an amount effective to treat or prevent one or more symptoms of one or more brain cancers or proliferative disorders and/or diseases, for example, reducing the amount or presence of one or more biomarkers associated with brain cancer.

### B. Dosage and Effective Amounts

Dosage and dosing regimens are dependent on the severity and location of the disorder or injury and/or methods of administration, and can be determined by those skilled in the art. A therapeutically effective amount of the glycosylated dendrimer composition used in the treatment of a proliferative disease or disorder in the brain is typically sufficient to reduce or alleviate one or more symptoms of brain cancer and/or proliferative disorder in the brain.

Preferably, the active agents do not target or otherwise modulate the activity or quantity of healthy cells not within or associated with the diseased/damaged tissue, or do so at a reduced level compared to cells associated with a disease or disorder such as a cancer and/or proliferative disorder. In this way, by-products and other side effects associated with the compositions are reduced. Therefore, in preferred embodiments, glycosylated dendrimer compositions are administered in an amount that leads to an improvement, or enhancement, function in an individual with a disease or disorder, such as a cancer and/or proliferative disorder.

The actual effective amounts of glycosylated dendrimer composition can vary according to factors including the specific agent administered, the particular composition formulated, the mode of administration, and the age, weight, condition of the subject being treated, as well as the route of administration and the disease or disorder. In some embodiments, dosage ranges suitable for use are between about 0.01 and about 100 mg/kg body weight, inclusive; between about 0.1 mg/kg and about 10 mg/kg, inclusive; between from about 0.5 mg and about 5 mg/kg body weight, inclusive. Generally, for intravenous injection or infusion, the dosage may be lower than for oral administration.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill in the art can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

Dosage forms of the pharmaceutical composition including the dendrimer compositions are also provided. “Dosage form” refers to the physical form of a dose of a therapeutic compound, such as a capsule or vial, intended to be administered to a patient. The term “dosage unit” refers to the amount of the therapeutic compounds to be administered to a patient in a single dose.

In general, the timing and frequency of administration will be adjusted to balance the efficacy of a given treatment or diagnostic schedule with the side effects of the given delivery system. Exemplary dosing frequencies include continuous infusion, single and multiple administrations such as hourly, daily, weekly, monthly, or yearly dosing.

In some embodiments, dosages are administered daily, biweekly, weekly, every two weeks or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

It will be understood by those of ordinary skill that a dosing regimen can be any length of time sufficient to treat the disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

In preferred embodiments, methods for treating or preventing one or more symptoms of a brain cancer and/or proliferative disorder in the brain of a subject in need thereof include administering to the subject a formulation including glycosylated dendrimers complexed to, covalently conjugated to, or having intra-molecularly dispersed or encapsulated therein one or more therapeutic or prophylactic agents in an amount effective to treat or prevent one or more symptoms of the brain cancer, or to reduce the viability or number of cancer cells in the subject. It will be understood by those of ordinary skill that a dosing regimen will be for an amount and for a length of time sufficient to treat a brain tumor to reduce size, metastasis, or rate of growth, or to alleviate one or more symptoms such as swelling, pain, or seizures. Physicians routinely determine the length and amounts of therapy to be administered. Typically, the glycosylated dendrimer conjugates including the one or more active agents are administered systemically, and are transported across the blood-brain-barrier (BBB) to enter the brain and are selectively taken up by TAMs and MDSCs in the region of a tumor. Typically, the glycosylated dendrimer conjugates accumulate within TAMs and MDSCs and deliver the active agents to these cells. The accumulation of glycosylated dendrimer conjugates in TAMs and MDSCs is up to 100 times that of un-glycosylated dendrimer complexes. Therefore, in some embodiments, the effective amount of active agent required for treatment or prevention of brain cancer is up to one hundredth (100 times less) than the amount required when using un-glycosylated dendrimer complexes, or the active agent alone, for example one quarter, one half, one fifth, one tenth, one twentieth, on thirtieth, one fortieth, one fiftieth, one sixtieth, one seventieth, one eightieth, one ninetieth, or one hundredth of the amount required when using un-glycosylated dendrimer complexes, or the active agent alone.

### C. Combination Therapies and Procedures

The glycosylated dendrimer compositions can be administered alone or in combination with one or more conventional therapies. In some embodiments, the conventional therapy includes administration of one or more of the compositions in combination with one or more additional active agents. The combination therapies can include administration of the active agents together in the same admixture, or in separate admixtures. Therefore, in some embodiments, the pharmaceutical composition more than one active agent. Such formulations typically include an effective amount of an agent targeting the site of treatment. The additional active agent(s) can have the same or different mechanisms of action. In some embodiments, the combination results in an additive effect on the treatment of the disease or condition. In some embodiments, the combinations result in a more than additive effect on the treatment of the disease or disorder.

In some embodiments, the glycosylated dendrimer composition is administered prior to, in conjunction with, subsequent to, or in alternation with, treatment with one or more additional therapies or procedures. In some embodiments, the additional therapy is performed between drug cycles or during a drug holiday that is part of the composition dosage regime. For example, in some embodiments, the additional therapy or procedure is surgery, a radiation therapy, or chemotherapy.

In some embodiments, the compositions and methods are used prior to or in conjunction with an immunotherapy such inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb).

Methods of adoptive T cell therapy are known in the art and used in clinical practice. Generally adoptive T cell therapy involves the isolation and ex vivo expansion of tumor specific T cells to achieve greater number of T cells than what could be obtained by vaccination alone. The tumor specific T cells are then infused into patients with cancer in an attempt to give their immune system the ability to overwhelm remaining tumor via T cells, which can attack and kill the cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In some embodiments, the T cells are taken directly from the patient's blood. Methods of priming and activating T cells in vitro for adaptive T cell cancer therapy are known in the art. See, for example, Wang, et al, Blood, 109(11):4865-4872 (2007) and Hervas-Stubbs, et al, J. Immunol., 189(7):3299-310 (2012).

Historically, adoptive T cell therapy strategies have largely focused on the infusion of tumor antigen specific cytotoxic T cells (CTL) which can directly kill tumor cells. However, CD4⁺T helper (Th) cells such as Thl, Th2, Tfh, Treg, and Th17 can also be used. Th can activate antigen-specific effector cells and recruit cells of the innate immune system such as macrophages and dendritic cells to assist in antigen presentation (APC), and antigen primed Th cells can directly activate tumor antigen-specific CTL. As a result of activating APC, antigen specific Th₁ have been implicated as the initiators of epitope or determinant spreading which is a broadening of immunity to other antigens in the tumor. The ability to elicit epitope spreading broadens the immune response to many potential antigens in the tumor and can lead to more efficient tumor cell kill due to the ability to mount a heterogeneic response. In this way, adoptive T cell therapy can used to stimulate endogenous immunity.

In some embodiments, the T cells express a chimeric antigen receptor (CARs, CAR T cells, or CARTs). Artificial T cell receptors are engineered receptors, which graft a particular specificity onto an immune effector cell. Typically, these receptors are used to graft the specificity of a monoclonal antibody onto a T cell and can be engineered to target virtually any tumor associated antigen. First generation CARs typically had the intracellular domain from the CD3 ζ-chain, which is the primary transmitter of signals from endogenous TCRs. Second generation CARs add intracellular signaling domains from various costimulatory protein receptors (e.g., CD28, 41BB, ICOS) to the cytoplasmic tail of the CAR to provide additional signals to the T cell, and third generation CARs combine multiple signaling domains, such as CD3z-CD28-41BB or CD3z-CD28-OX40, to further enhance effectiveness.

In some embodiments, the compositions and methods are used prior to or in conjunction with a cancer vaccine, for example, a dendritic cell cancer vaccine. Vaccination typically includes administering a subject an antigen (e.g., a cancer antigen) together with an adjuvant to elicit therapeutic T cells in vivo. In some embodiments, the cancer vaccine is a dendritic cell cancer vaccine in which the antigen delivered by dendritic cells primed ex vivo to present the cancer antigen. Examples include PROVENGE® (sipuleucel-T), which is a dendritic cell-based vaccine for the treatment of prostate cancer (Ledford, et al., Nature, 519, 17-18 (5 Mar. 2015). Such vaccines and other compositions and methods for immunotherapy are reviewed in Palucka, et al., Nature Reviews Cancer, 12, 265-277 (April 2012).

The additional therapy or procedure can be simultaneous or sequential with the administration of the dendrimer composition. In some embodiments, the additional therapy is performed between drug cycles or during a drug holiday that is part of the composition dosage regime.

In some embodiments, the compositions and methods are used prior to or in conjunction, subsequent to, or in alternation with treatment with one or more additional therapies or procedures. In some embodiments, the compositions and methods are used prior to or in conjunction with surgical removal of tumors, for example, in preventing primary tumor metastasis. In some embodiments, the compositions and methods are used to enhance body's own anti-tumor immune functions.

### D. Controls

The therapeutic result of the glycosylated dendrimer complex compositions including one or more active agents can be compared to a control. Suitable controls are known in the art and include, for example, an untreated subject, or a placebo-treated subject. A typical control is a comparison of a condition or symptom of a subject prior to and after administration of the targeted agent. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art. In some embodiments, an untreated control subject suffers from the same disease or condition as the treated subject.

In some embodiments, a control includes an equivalent amount of active agent delivered alone, or bound to OH-dendrimers in the absence of conjugated glucose.

### V. Kits

The compositions of glycosylated dendrimer can be packaged in kit. The kit can include a single dose or a plurality of doses of a composition including one or more active agents, encapsulated in, associated with, or conjugated to a glycosylated dendrimer (e.g., a glucosylated dendrimer), and instructions for administering the compositions. Specifically, the instructions direct that an effective amount of the glycosylated dendrimer composition be administered to an individual with a particular disease/disorder as indicated. The composition can be formulated as described above with reference to a particular treatment method and can be packaged in any convenient manner.

The present invention will be further understood by reference to the following non-limiting examples.

### EXAMPLES

### Example 1: Synthesis and Characterization of Glucosylated Dendrimers

To establish whether dendrimer-mediated targeting strategies can be enhanced through the addition of carbohydrates, hydroxyl-terminated polyamidoamine (PAMAM) dendrimers were conjugated to glucose, galactose, or mannose moieties, to determine their efficacy as ligands to leverage altered metabolism in cancer and immune targeting. Using a highly facile click chemistry approach, the surfaces of dendrimers were modified with glucose, mannose, or galactose moieties in a well-defined manner, to target upregulated sugar transporters in the context of glioblastoma.

### Materials and Methods

### Reagents

Pharma grade generation 4 hydroxyl PAMAM dendrimer with ethylenediamine-core (64 hydroxyl terminal-groups, D-OH) was purchased from Dendritech in the form of methanolic solution. Methanol was removed under vacuo prior to use. Azido-PEG-4-amine was purchased from Broadpharm. 5-Hexynoic acid, 1-ethyl-3-(3-dimethylaminopropyl)carbodiimide (EDC), 4(dimethylamine)pyridine (DMAP), copper sulfate pentahydrate, sodium ascorbate was purchased from Sigma Aldrich US. Cy5 NHS ester was purchased from GE healthcare. All the solvents were purchased from Sigma and were used as received. Deuterated solvents for NMR were purchased from Sigma. The experiments were conducted in standard oven-dried glassware. The click reactions with sugar-azides were performed in the microwave reactor using microwave safe vials.

### Synthetic protocols

β-D-Glucose-PEG4-azide, β-D-Galactose-PEG4-azide, and α-D-Mannose-PEG4-azide were synthesized using previously published protocol and were characterized using 1H NMR spectroscopy (Sharma, et al., Journal of Controlled Release, 283 (2018) 175-189; Sharma, et al., Chemical Communications, 50 (2014) 13300-13303.)

### Synthesis of Compound 3 (D-Hexyne)

In an over-dried flask, compound 2 (5 g, 0.35 mmoles) was dissolved in anhydrous N, N dimethylformamide (DMF, 20 mL). The solution was stirred and 5-hexynoic acid (980 mg. 8.75 mmoles) was added followed by the addition of DMAP (512 mg, 4.) and EDC (1.67 g, 8.75 mmoles). The stirring was continued at room temperature for 24 hours. The reaction was then diluted with DMF (100 mL) and transferred to the dialysis membrane (1000 Da cut off). The dialysis was first performed against DMF, followed by water until all the DMF was exchanged by the water. The water solution was then lyophilized to obtain the product as white solid (80% yield). ¹H NMR (500 MHz, DMSO) δ 8.09-7.74 (m, D-internal amide H), 4.71 (bs, D-OH), 4.02 (t, J=5.5 Hz, ester —CH₂), 3.44-3.24 (m, dendrimer-CH₂), 3.10-3.03 (m, dendrimer-CH₂ and linker H), 2.81-2.57 (m, dendrimer-CH₂ and linker H), 2.48-2.34 (m, dendrimer-CH₂), 2.27-2.12 (m, dendrimer-CH₂), 1.71 (t, linker —CH₂).

### Synthesis of Compound 5 (NH₂-D-Hexyne)

In an over-dried flask, compound 3 (5 g. 0.316 mmoles) was dissolved in anhydrous N, N dimethylformamide (DMF, 10 mL). The solution was stirred, and azido-PEG-4-amine (323 mg, 1.26 mmoles) was added dissolved in DMF (1 mL). To the stirring reaction mixture, catalytic amount of CuSO4.5H₂O (40 mg, 0.158 mmoles) dissolved in water (1 mL) was added followed by the addition of the catalytic amount of sodium ascorbate (31 mg, 0.158 mmoles) dissolved in water (1 mL). The reaction mixture was stirred at 40° C. overnight. The reaction was then brought to room temperature and diluted with water (100 mL). A solution of ethylene-diaminetetracetic acid (EDTA, 1 mL) was added and dialysis was performed against water. The water solution was then lyophilized to obtain the product as white solid (82% yield).

¹H NMR (500 MHz, DMSO) δ 8.15-7.68 (m, D-internal amide H & PEG traizole H), 4.47 (t, triazole —CH₂), 4.01 (t, ester —CH₂), 3.80 (t, triazole —CH₂), 3.57-3.22 (m, PEG H, dendrimer-CH₂), 3.19-3.00 (m, dendrimer-CH₂), 2.82-2.57 (m, dendrimer-CH₂ and linker H), 2.43-2.32 (m, dendrimer-CH₂), 2.30-2.03 (m, dendrimer-CH₂), 1.83 (m, linker H), 1.69 (t, linker H). 1H NMR (500 MHz, MeOD) δ 7.76 (s, PEG traizole H), 4.46 (t, triazole —CH₂), 4.05 (t, ester —CH₂), 3.79 (t, triazole —CH₂), 3.63-2.97 (m, PEG H, dendrimer-CH₂), 2.84-2.58 (m, dendrimer-CH₂ and linker H), 2.41-2.05 (m, dendrimer-CH₂ and linker H), 1.87 (m, linker H), 1.70 (t, linker H).

### Synthesis of Compound 6 (D-Sugar)

To a stirring solution of compound 5 (1 g, 0.06 mmoles) in DMF (4 mL) in a microwave-safe reaction vial, a solution of sugar azide (367.9 mg, 0.96 mmoles) in DMF (1 mL) was added. This was followed by the addition of catalytic amount of CuSO4.5H₂O (15 mg, 0.06 mmoles) dissolved in water (1 mL) and sodium ascorbate (12 mg, 0.06 mmoles) dissolved in water (1 mL). The reaction was stirred in a microwave reactor (Biotage) at 50° C. for 8 hours. The reaction was then brought to room temperature and diluted with water (100 mL). A solution of ethylene diaminetetracetic acid (EDTA, 1 mL) was added and dialysis was performed against water. The water solution was then lyophilized to obtain the product as white solid (80-90% yield).

### D-Glucose, 6a

¹H NMR (500 MHz, DMSO) δ 8.39-7.49 (m, dendrimer internal amide H+Triazole H), 4.50-4.42 (m, triazole-CH2), 4.15 (d, J=6.3 Hz, anomeric H glucose), 4.05-3.98 (m, linker CH₂+glucose), 3.90-3.75 (m, glucose H), 3.69-2.85 (m, dendrimer+PEG+glucose H), 2.80-2.56 (m, dendrimer H), 2.40-2.15 (m, dendrimer H), 1.84 (s, hexynoic linker CH₂).

¹H NMR (500 MHz, D20) δ 7.96-7.84 (m, triazole glucose+PEG H), 4.63-4.55 (m, triazole-CH2), 4.48 (d, J=7.1 Hz, glucose anomeric H), 4.08-4.02 (m, glucose H), 4.05-3.88 (m, linker CH₂+glucose), 3.86-3.23 (m, dendrimer+PEG+glucose H), 3.08-2.29 (m, dendrimer H), 2.02-1.89 (m, hexynoic linker CH₂).

### D-Galactose, 6b

¹H NMR (500 MHz, DMSO) δ 8.18-7.58 (m, dendrimer internal amide H+triazole H), 4.46 (s, triazole-CH₂), 4.13-4.02 (m, galactose CH₂), 4.03-3.98 (m, dendrimer CH₂+galactose H), 3.85-3.77 (galactose H), 3.65-3.35 (m, dendrimer+PEG H), 3.34-3.17 (dendrimer H), 3.14-3.05 (dendrimer H), 2.91-2.88 (m, dendrimer H), 2.75-2.58 (m, dendrimer H), 2.44-2.05 (m, dendrimer H), 1.83 (s, hexynoic acid linker CH₂).

¹H NMR (500 MHz, D₂O) δ 7.93 (br s, PEG-4 triazole, 3H), 7.86 (br s, galactose triazole, 13H), 4.60-4.55 (m, CH2-triazole, 30H), 4.42 (d, J=7.4 Hz, galactose anomeric H), 4.16-4.12 (m, galactose H), 4.07-4.04 (m, galactose H), 3.96-3.88 (m, dendrimer-galactose H), 3.85-3.48 (m, dendrimer-galactose H), 3.47-3.43 (m, galactose H), 3.35-3.26 (m, dendrimer H), 3.02-2.99 (m, dendrimer H), 2.97-2.58 (m, dendrimerH), 2.44 (s, dendrimerH), 1.99-1.90 (m, CH2-hexynoic acid linker).

### D-Mannose, 6c

¹H NMR (500 MHz, DMSO) δ 8.15-7.75 (m, dendrimer internal amide H+triazole H), 4.64-4.61 (m, mannose anomeric H), 4.49-4.44 (m, triazole-CH2), 4.04-3.93 (m, linker CH₂), 3.7-3.3 (m, dendrimer+glucose+PEG H), 3.71-3.19 (m, dendrimer H), 3.18-3.00 (m, dendrimer H), 2.66-2.56 (m, dendrimer H), 2.45-2.04 (m, dendrimer H), 1.87-1.79 (m, hexynoic linker CH₂).

¹H NMR (500 MHz, D₂O) δ 7.93 br (s, PEG triazole H), 7.86 (br s, mannose triazole H), 4.61-4.55 (m, triazole-CH₂), 4.40 (d, J=7.4 Hz, mannose anomeric H), 4.18-4.02 (m, mannose H), 3.97-3.88 (m, linker CH₂+mannose H), 3.85-3.41 (m, dendrimer+mannose+PEG H), 3.37-3.27 (m, dendrimer H), 3.30-2.30 (m, dendrimer H), 2.50-2.35 (m, dendrimer H), 1.98-1.90 (m, hexynoic linker CH₂).

Synthesis of compound 7 (Cy5-D-Sugar): In an over-dried flask, compound 6 (50 mg. 0.0023 mmoles) was dissolved in anhydrous N, N dimethylformamide (DMF, 4 mL). The solution was stirred, and the pH was brought to 7.4 using DIPEA. This was followed by the addition of Cy5-NHS ester (5 mg, 0.007 mmoles). The reaction was stirred at room temperature for 12 hours, protected from light. The reaction was then diluted with DMF (50 mL) and transferred to the dialysis membrane (1000 Da cut off). The dialysis was first performed against DMF, followed by water dialysis until all the DMF is exchanged by the water. The water solution was then lyophilized to obtain the product as blue solid which was further purified on G-25 sephadex column (65-75% yield).

¹H NMR (Cy5-D-Glucose, 7a): 1H NMR (500 MHz, DMSO) δ 8.42-8.33 (m, Cy5 H), 8.20-7.68 (m, dendrimer internal amide H+triazole H), 7.68-7.61 (m, Cy5 H), 7.35-7.30 (m, Cy5 H), 6.35-6.27 (m, Cy5 H), 5.03-4.87 (m, glucose H), 4.80-4.65 (m, dendrimer OH), 4.49 (m, triazole-CH₂), 4.20-3.92 (m, glucose+dendrimer linker CH₂), 3.90-3.74 (m, glucose H), 3.69-2.89 (m, dendrimer+glucose+PEG H), 2.80-2.60 (m, dendrimer H), 2.46-2.10 (m, dendrimer H), 1.89-1.79 (m, dendrimer H), 1.71-1.14 (m, Cy5 H and linker H). HPLC: Purity >99%, Retention time: 12.83 min.

¹H NMR (Cy5-D-Galactose, 7b): 1H NMR (500 MHz, DMSO) δ 8.36 (t, J=12.7 Hz, Cy5 H), 8.12-7.72 (m, dendrimer internal amide H+triazole H), 7.66-7.58 (m, Cy5 H), 7.36-7.26 (m, Cy5 H), 6.62-6.53 (m, Cy5 H), 4.87-4.40 (m, galactose H), 4.36 (m, triazole-CH₂), 4.19-3.71 (m, glucose+dendrimer linker CH₂), 3.66-2.96 (m, dendrimer+galactose+PEG H), 2.78-2.55 (m, dendrimer H), 2.44-2.05 (m, dendrimer H), 1.89-1.74 (m, dendrimer H), 1.73-1.12 (m, Cy5 H and linker H); HPLC: Purity >99%, Retention time: 13.02 min.

¹H NMR (Cy5-D-Mannose, 7c): 1H NMR (500 MHz, DMSO) δ 8.37 (t, J=12.1 Hz, Cy5 H), 8.15-7.75 (m, dendrimer internal amide H+triazole H), 7.67-7.61 (m, Cy5 H), 7.33-7.27 (m, CY5 H), 6.33-6.26 (m, CY5 H), 4.85-4.55 (m, mannose H+OH+dendrimer OH), 4.50-4.38 (m, triazole-CH₂), 4.17-3.93 (m, m, linker CH₂+mannose H), 3.75 (m, mannose H), 3.71-3.23 (m, dendrimer+mannose+PEG H), 3.16-3.03 (m, dendrimer H), 2.65-2.55 (m, dendrimer H), 2.47-2.07 (m, dendrimer H), 1.84 (m, m, hexynoic linker CH2), 1.72-1.10 (m, Cy5 H and linker H); HPLC: Purity >99%, Retention time: 12.97 min.

### Instrumentation for Characterization of Intermediates and Dendrimer Conjugates

The structures of intermediates and dendrimer conjugates were analyzed using proton nuclear magnetic resonance (¹H NMR) spectroscopy. NMR spectra were recorded on a Bruker spectrometer (500 MHz) at ambient temperatures. The NMR data is presented as chemical shift values (6 ppm) and multiplicity. The chemical shifts of the residual protic solvent such as CDCl₃ (1H, δ 7.27 ppm), D₂O (1H, δ 4.79 ppm); and DMSO-d6 (1H, δ2.50 ppm) were used for chemical shifts calibration. The purity of the final dendrimer conjugates was evaluated using high pressure liquid chromatography (HPLC). HPLC was run on a Waters Corporation system equipped with 2998 photodiode array detector and a 2475 multi k fluorescence detector. The instrument had an in-Line degasser, and a 1525 binary pump. The chromatograms were analyzed using Waters Empower 2 Software. The dendrimer samples were run through a C18 Waters column (C18 symmetry 300, 5 μm, 4.6×250 mm) maintaining the flowrate at 1.0 m/min. A gradient flow method was used using 0.1% TFA and 5% acetonitrile in water (A) and 0.1% TFA in acetonitrile (B). The method started from a gradient 90:10 (A:B), stayed at 90:10 (A:B) for 3 minutes, gradually increasing to 50:50 (A:B) at 16 minutes, stayed at 50:50 (A:B) at 25 minutes, and finally returned to 90:10 (A:B) at 35 minutes. The chromatogram were monitored at wavelengths 210 (dendrimer absorption) and 650 nm (Cy5 absorption). The particle size distribution and zeta potential distribution of dendrimer conjugates were measured via Dynamic light scattering on a Malvern Zetasizer Nano ZS instrument using previously our previously published protocols. (Sharma, et al., Journal of Controlled Release, 283 (2018) 175-189.)

### Results

Glycosylation of nanoparticles has attracted significant interest in the development of targeted drug delivery systems due to their specific ligand-receptor recognition. Since sugars are natural targeting ligands and are an integral part of several biological processes in human body, their incorporation into nanoparticles provides stealth without compromising their cellular uptake, biocompatibility, non-immunogenicity and enhance their blood circulation time and enzymatic stability in the serum (Roy, et al., Chemical Society Reviews, 44 (2015) 3924-3941; Zhang, et al., Journal of controlled release, 219 (2015) 355-368). Moreover, sugar units are synthetically appealing for functionalization of nanocarriers and can be easily modified specifically at the anomeric position for the attachment of linkers. To assess how surface decoration with sugars impacts dendrimer in vivo transport and targeting properties in glioblastoma model, generation 4 hydroxyl-terminated PAMAM dendrimers were conjugated to β-D-glucose, β-D-galactose, or α-D-mannose via click chemistry approach (see FIG. 1A).

In recent years, click chemistry has been a powerful tool to create libraries of small molecules, synthesis of complex macromolecules and for the surface conjugation of polymers and dendrimers. Sugars were modified with a short PEG linker to reduce steric hindrance to receptor interactions. This sugar linker was then attached to the dendrimer surface via copper catalyzed azide-alkyne click (CuAAC) reaction to produce sugar dendrimer conjugates. The synthesis began with the construction of clickable glucose, galactose & mannose-azides (1a,b,c) using established protocols according to the scheme in FIG. 1B. On the other hand, the alkyne-terminating clickable dendrimer was synthesized by partial modification of 14-16 OH groups on the surface of dendrimer (D-OH, 2). These hydroxyl groups were esterified with 5-hexynoic acid using EDC/DMAP coupling chemistry to afford D-hexyne (3). 3-4 arms of alkyne groups on D-hexyne were clicked with azido-PEG-4amine (4) to generate a trifunctional dendrimer (NH₂-D-hexyne, 5) with ˜12 alkyne and ˜3-4 amine functional groups. The alkyne terminal groups were meant to participate in CuAAC reaction with azide terminating sugars while amine surface groups will be utilized for the attachment of near infra-red dye cyanine 5 (Cy5) for imaging purpose. The dendrimer (5) was reacted with sugar-azides (1) (β-D-Glucose-PEG4-azide, β-D-Galactose-PEG4-azide, and α-D-Mannose-PEG4-azide) using CuAAC reaction in the presence of catalytic amounts of copper sulphate pentahydrate and sodium ascorbate to yield corresponding dendrimer-sugar conjugates (D-Sugar, 6). The dendrimer-sugar conjugates were purified in the presence of ethylenediaminetetraacetic acid (EDTA) using tangential flow filtration to remove excess of reagents and the traces of copper. Use of click chemistry enabled a precise control of sugar payload on the dendrimer. Only 12-14 hydroxyl groups were utilized on the dendrimer surface for the attachment of sugar moieties to maintain the inherent brain tumor targeting properties of hydroxyl PAMAM dendrimers (nearly neutral surface charge; ˜5 nm) while exploring the effect of various sugars as targeting ligands. It has been previously shown that the surface modification up to 20 wt % does not alter the properties of dendrimers to target neuroinflammation and brain tumor. The amine groups in each of the D-Sugar conjugates were reacted with Cy5-mono-NHS ester at pH7.5-8 to obtain fluorescently labeled Cy5-D-Sugars [Cy5-D-Glucose (7a); Cy5-D-Galactose (7b); and Cy5-D-Mannose (7c)].

### Characterization and Chemical Validation of Dendrimer Sugar Conjugates

Throughout the synthesis of fluorescently labeled D-Sugar conjugates, the structures were analyzed at every step via ¹H NMR spectroscopy. Comparison of spectra from ¹H NMR intermediates, D-Sugars and fluorescently labeled dendrimer conjugates showed the appearance of characteristic signals corresponding to sugar protons and Cy5 protons along with the parent dendrimer protons. The integration comparison of sugar and Cy5 protons to internal amide protons from dendrimer was utilized to calculate the number of conjugated ligands.

The synthesis of D-Hexyne (3) was confirmed by the appearance of ester methylene proton at δ 4.0 ppm and a methylene proton from hexyne linker at δ1.7 ppm. The number of hexyne linkers conjugated in the surface of the dendrimer were calculated by comparing the integration of dendrimer internal amide protons to ester methylene protons, which revealed ˜14-16 linkers were attached. HPLC showed a shift in retention time from D-OH at 9.5 min to D-Hexyne at 13.4 min. The success of the partial click with amine terminating PEG linker was analyzed by the appearance of triazole methylene protons and the triazole proton at δ 7.76 ppm when NMR was taken in methanol to exchange dendrimer amide protons. The presence of a few amine groups resulted in a shift in retention time on HPLC at 9.6 min. The NMR spectra of resulting D-Sugars after click reaction with sugar-azides clearly showed the presence of proton signals from glucose, galactose, and mannose along with other dendrimer signals. To confirm the success of click reaction, the NMRs were also taken in D₂O. The deuterium exchange resulted in the disappearance of internal amide protons revealing two distinct peaks corresponding to the triazole protons from two different click reactions. The resulting dendrimer-sugar conjugates were highly pure (>99% purity) as analyzed by HPLC; comparison of parent hydroxyl dendrimer (D-OH, RT: 9.5 min), alkyne-terminating dendrimer (D-Hexyne, RT: 13.4 min), trifunctional dendrimer (NH₂-D-Hexyne, RT: 9.6 min), and sugar modified dendrimers (D-Glucose, RT: 11.3 min; D-Galactose, RT: 11.4 min; and D-Mannose, RT: 11.7 min). All the intermediates and sugar conjugates have >99% purity. Hydrodynamic diameter measurements of D-Sugars via dynamic light scattering. The dendrimer conjugates show a slight increase in size from D-OH (˜4 nm). Representation of zeta potential distribution measurements of D-Sugars showing nearly neutral zeta potential.

Remarkably tight loading of ˜12 molecules of sugars in each conjugate was evident from ¹H NMR and HPLC showing click reaction as a stupendous tool for the ligation on the surface of macromolecules. Upon Cy5 labeling, appearance of Cy5 protons in aromatic, allyl and aliphatic regions were evident from the NMR spectra and confirmed the attachment of 1-2 molecules of Cy5 in all three conjugates. The HPLC purity at 650 nm (Cy5 absorbance) showed highly pure dendrimers with purity greater than 99%. The tumor targeting potential of hydroxyl PAMAM dendrimers is due to their small size and nearly neutral zeta potential. The hydrodynamic diameter and zeta potential of D-sugars were further measured to evaluate the effect of sugar conjugation. The size distribution by number and zeta potential distribution were characterized by dynamic light scattering (DLS). The sugar conjugation resulted in a slight increase in the size from D-OH (˜4 nm) to −5 nm for D-Sugars. The sizes of D-GLU, D-GAL, and D-MAN were 4.79 nm, PDI: 0.48; 4.53 nm, PDI: 0.49; and 4.97 nm, PDI: 0.52 respectively. The zeta potential was nearly neutral for all three dendrimer-sugar conjugates in the range from 7-10 mV.

### Example 2: D-GLU Exhibits Increased Tumor Accumulation

### Materials and Methods

### Reagents

Bovine serum albumin (BSA), 4-[[[[4-(1,1-Dimethylethyl)phenyl]sulfonyl]amino]methyl]-N-3-pyridinylbenzamide (STF-31), and α-lactose were obtained from Sigma Aldrich (St. Louis, MO, USA). Dulbecco's modified eagle medium (DMEM), Roswell Park Memorial Institute (RPMI) media, L-glutamine, fetal bovine serum (FBS), penicillin-streptomycin (P/S) antibiotic, 0.25% trypsin-EDTA, normal goat serum (NGS), goat anti-rabbit Alexafluor 488, and (3-(4,5-dimethylthiazol-2-yl)-2,5-diphenyl tetrazolium bromide) MTT reagent were purchased from Invitrogen (Carlsbad, CA, USA). Tris-buffered saline (TBS) and phosphate buffered saline (PBS) were purchased from Corning (Corning, NY, USA). Ionized calcium binding adaptor molecule (Ibal) primary antibody was purchased from Wako Pure Chemical Corporation (Tokyo, Japan). NucBlue cell stain (DAPI) was purchased from Cell Signaling (Danvers, MA, USA). Methanol was purchased from ThermoFisher (Waltham, MA, USA). Recombinant murine interleukin 4 (IL4) was obtained from Peprotech Inc.

### Tumor Inoculations

GL261 murine microglia were obtained from the DTP/DCTD/NCI Tumor Repository (National Cancer Institute, Frederick, MA, USA). Cells were cultured in RPMI supplemented with 10% FBS, 1% P/S, and 1% L-glutamine and maintained at 37° C. and 5% CO₂ atmosphere. All animals were housed at Johns Hopkins University animal facilities and given free access to food and water. Experiments performed were approved by the Johns Hopkins Institutional Animal Care and Use Committee. Male and female C57BL/6 mice were obtained from Jackson Laboratory Company (Bar Harbor, ME, USA). Mice 6-8 weeks old were inoculated with glioblastoma tumors via intracranial injection of GL261 cells. Mice were anesthetized with a ketamine/xylazine cocktail for surgeries. An incision was created in the center of the scalp. A burr hole was drilled at 1 mm posterior to the bregma and 2 mm lateral to the midline for injection into the striatum in the right hemisphere. A 2 μL injection of 100,000 GL261 cells were injected to a depth of 2.5 mm over 10 min with a stereotactic frame and automated syringe pump (Stoelting Co., Wood Dale, IL, USA). The syringe was withdrawn, and the incision sutured (Ethicon Inc., Somerville, NJ, USA).

### Immunohistochemistry and Confocal Microscopy

On day 14 post-inoculation, glioblastoma bearing mice were intravenously injected with unmodified hydroxyl-terminated dendrimers, dendrimer-glucose, dendrimer-mannose, or dendrimer-galactose conjugates at 55 mg/kg on the whole conjugate basis. At this time point, tumors exhibit an average mass of 34.0±6 mg. 24 hours after injection, brains, livers, and kidneys were collected and fixed overnight in 4% formalin solution (n=2 per construct). Organs were then passed through a sucrose gradient (10%, 20%, then 30% sucrose in PBS overnight each) to remove formalin. Brains were sectioned axially into 30 μm slices with a Leica CM 1905 cryostat (Wetzlar, Germany). Brains were stained for DAPI to visualize nuclei and Ibal (1:200) to visualize tumor-associated macrophages. Kidneys were stained for GFAP (1:500) to visualize tubules. Livers were stained for serum albumin (1:1000, Abcam) to visualize hepatocytes or SE-1 (1:200, Novus Biologicals) to visualize sinusoidal endothelial cells. Images were acquired on a Zeiss LSM710 confocal microscope (Hertfordshire, UK). Image capture settings and processing adjustments were kept constant across all compared images with different dendrimer types.

### Dendrimer Tissue Extraction and Quantification

At specified time points (1, 4, 24 hours) after administration, animals were euthanized and perfused with saline to remove residual blood in organs. Organs and plasma were collected and snap frozen in liquid nitrogen (n=5 per construct). To assess tumor targeting, tumors and healthy brain tissue from the contralateral hemisphere were dissected out. Tissues were dissected (100 mg for livers and kidneys, 50 mg for hearts and lungs, 20 mg for spleens) for dendrimer extraction. Tumors were homogenized and quantified whole and presented as μg/g tissue to normalize for tumor size. Tissue samples were homogenized in methanol at 100 μL per 100 mg with stainless steel homogenization beads (Next Advance, Troy, NY, USA), followed by sonication for 15 minutes. Samples were spun down at 12,000 rpm and supernatants collected for analysis. Plasma samples were diluted 5× in PBS and filtered through a 0.2 μm filter. For quantification, dendrimer solutions were read on a Shimadzu RF-3501 spectro-fluoro-photometer (Kyoto, Japan). Control samples of brain tumors without dendrimer administration were used to correct for tissue background fluorescence. The wavelengths used for the Cy5 labeled conjugates were excitation 645 nm and emission 662 nm. Calibration curves for each dendrimer of known concentrations were created to convert measured intensities to tissue concentrations. Tumor specificity was calculated as the ratio of dendrimer content within the tumor divided by content in the contralateral hemisphere. To explore dendrimer-glucose blood brain barrier penetration, healthy mice were intravenously injected with dendrimer, collected 24 hours later, and processed as described above.

### In Vitro Dendrimer Uptake Experiments

BV2 murine microglia and GL261 murine glioblastoma cells were maintained in incubators at 37° C. and 5% CO₂ atmosphere. BV2 cells were grown in DMEM supplemented with 10% FBS and 1% P/S. GL261 cells were grown in RPMI supplemented with 10% FBS, 1% P/S, and 1% L-glutamine. Treatments were performed in half serum media (5% FBS). Cell viabilities for each dendrimer conjugate were measured using MTT assay as per manufacturer's procedure.

To explore dendrimer-glucose cell interactions, BV2 microglia were treated with 50 μg/mL unmodified or glucose conjugated dendrimers. For blocking experiments, cells were exposed to STF-31 at 10 μM for 24 hours to block GLUT-1 transporters, followed by incubation with dendrimers for 6 hours. For cellular internalization, BV2 cells were incubated with unmodified and glucose dendrimers for 24 hours in the absence or presence of IL4. Cells were then collected in methanol, dendrimer extracted, and quantified on the spectrofluorophotometer as described above for tissues. For dendrimer uptake in GL261 cells, tumor cells were incubated with 10 μg/mL of each dendrimer for 24 hours. Cells were then washed, collected, and homogenized as described. To explore dendrimer-galactose cell interactions, GL261 glioblastoma cells were incubated with unmodified or galactose dendrimers for 24 hours in the presence or absence of α-lactose at 100 μM to block galectins. Membrane and cytosolic fractions were separated using the Mem-PER Plus protein extraction kit (ThermoFisher) and measured using the spectrofluorophotometer.

### Statistical Analyses

Statistical analyses and graphs were created using GraphPad Prism v8.0 software (San Diego, CA, USA). All error bars presented in figures show mean±standard errors. Statistical significances between dendrimer types in biodistribution were calculated using two-way ANOVAs. Differences between groups in in vitro experiments were performed using Student's t-tests. ill, NJ, USA).

### Results

Glucose modification significantly enhanced targeting of tumor-associated macrophages (TAMs) and microglia by increasing brain penetration and cellular internalization, while galactose modification shifted targeting away from TAMs towards galectins on glioblastoma tumor cells. Mannose modification did not alter TAMs and microglia targeting of these dendrimers but did alter their kinetics of accumulation within the GBM tumor. The whole body biodistribution was largely similar between the systems. These results demonstrate that dendrimers are versatile delivery vehicles that can be modified to tailor their targeting for the treatment of glioblastoma and other cancers.

### Localization of Sugar Dendrimer Conjugates In Vivo in Orthotopic Brain Tumors

To assess impact of sugar dendrimer conjugation on in vivo trafficking, glioblastoma brain tumor-bearing mice were injected intravenously with fluorescently labeled (unmodified Cy5 dendrimer (D-OH), Cy5-D-glucose-(D-GLU), Cy5-D-mannose-(D-MAN), or Cy5-D-galactose (D-GAL) dendrimers. Dendrimers were administered 14 days after tumor inoculation, and brains were collected for imaging 24 hours post-injection. Brains were then stained with DAPI to visualize cell nuclei and lectin (Lycopersicon Esculentum lectin) to label TAMs and microglia.

Hydroxyl terminated dendrimers (D-OH) can overcome brain and solid tumor barriers to selectively localize within TAMs and activated microglia, as detected in photomicrographs; Glioblastoma brain tumor-bearing mice were injected with various dendrimers on day 14 after inoculation. Brains were collected 24 hours after administration, fixed, and stained with lectin to label tumor-associated macrophages/microglia (TAMs, green) and DAPI to label nuclei (blue) for confocal microscopy to visualize dendrimer (red) localization. Unmodified dendrimer (D-OH) localizes to TAMs within the tumor upon systemic administration. Glucose-(D-GLU) and mannose-conjugated dendrimers (D-MAN) maintain the TAMs localization of D-OH. Galactose-conjugated dendrimer (D-GAL) exhibits some TAMs localization, with additional signal observed in the extracellular space.

D-OH exhibited perinuclear signal pattern within these cells. D-GLU and D-MAN exhibited the same TAMs and microglia targeted signal, indicating that the dendrimer transport properties are preserved, upon sugar modification. Mannose receptors (CD206) are highly upregulated on anti-inflammatory, pro-tumor macrophages and have been leveraged in TAMs targeting platforms. Therefore, the TAMs targeting of D-MAN was expected. However, D-GLU also exhibited the same TAMs and microglia targeting, which was a surprising result. Glucose has been explored to bring therapies directly to cancer cells by leveraging the altered metabolism exhibited by cancer cells. However, D-GLU exhibited the same TAMs and microglia targeting as D-OH, indicating that glucose moieties do not overcome dendrimer affinity for TAMs and microglia. In addition, macrophages have also been shown to exhibit upregulated glucose transporters in the context of inflammation. D-GAL exhibited highly distinct signal pattern separate from D-OH and other sugar modified dendrimers

While some signal was observed in TAMs and microglia, D-GAL primarily exhibited highly punctated signal pattern in the tumor microenvironment. This signal pattern likely arises from D-GAL interactions with galectins on the surface of cancer cells. Galectins are highly overexpressed in many cancers, including glioblastoma, and have been shown to regulate cell interactions with the extracellular matrix to mediate cancer cell invasion and metastasis.

Sugars may also interact with receptors on other cell types in peripheral organs. To explore off-target interactions after systemic administration, unmodified and sugar dendrimers were injected intravenously into brain tumor bearing mice. 24 hours after injection, kidneys and livers were collected and stained for confocal imaging. Kidneys were stained with DAPI to label nuclei and GFAP to label proximal tubules. Livers were stained with DAPI to label nuclei and serum albumin to label hepatocytes or SE1 to label sinusoidal endothelial cells. D-OH exhibited high localization within renal proximal tubules, consistent with nanoparticle clearance via kidney fenestrations in this size range. D-GLU, D-MAN, and D-GAL exhibited similar signal localization within the tubules, indicating that the primary clearance route of dendrimers is not altered with sugar surface decoration. In the kidneys, D-OH exhibits minimal signal lining the portal veins, indicating negligible liver accumulation. In contrast, D-GAL exhibits broad signal throughout the liver targeted to hepatocytes. This is consistent with established reports that hepatocytes exhibited high expression of asialoglycoprotein receptors, which bind with galactose. This hepatocyte targeting has implications for targeting liver diseases and has been used for targeted delivery in liver diseases and hepatic cancers. D-GLU and D-MAN exhibited signal localized to within sinusoidal endothelia. This indicates that in addition to renal clearance, D-GLU and D-MAN may also be experiencing clearance from the body via liver filtration. This is consistent with previous reports where mannose receptor mediates uptake by sinusoidal endothelial cells for clearance. Surprisingly, despite receptors for these sugars being implicated in Kupffer cell internalization, no uptake in Kupffer cells was observed with these sugar dendrimers, indicating that the risk of off-target immune modulation with these sugar dendrimers in the liver is minimal.

### Quantification of Tumor Accumulation by Sugar Dendrimer Conjugates

To quantify tumor accumulation, brain tumor bearing mice were intravenously injected with unmodified or sugar-modified dendrimers on day 14 after tumor inoculation. Brains and organs were then collected at specified time points, extracted for dendrimers, and quantified with fluorescence quantification.

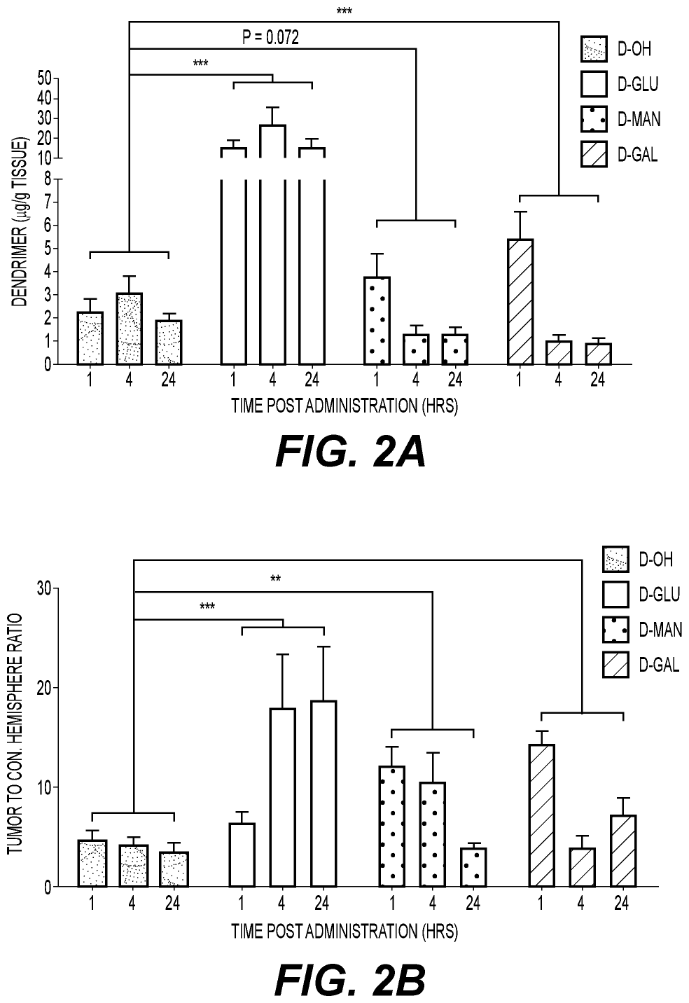

Glioblastoma brain tumor-bearing mice were injected intravenously with various dendrimers on day 14 after tumor inoculation. Brains were collected 24 hours after injection, homogenized, and dendrimer content was measured via fluorescence spectrometry. FIG. 2A shows brain tumor-bearing mice injected with glucose-(D-GLU), mannose (D-MAN), and galactose-conjugated dendrimers (D-GAL) exhibit significantly greater tumor accumulation than unmodified dendrimers (D-OH). *** p<0.001. FIG. 2B Sugar-conjugated dendrimers exhibit significantly greater specificity for the tumor compared to the contralateral hemisphere than unmodified dendrimers. ** p<0.01, *** p<0.001.

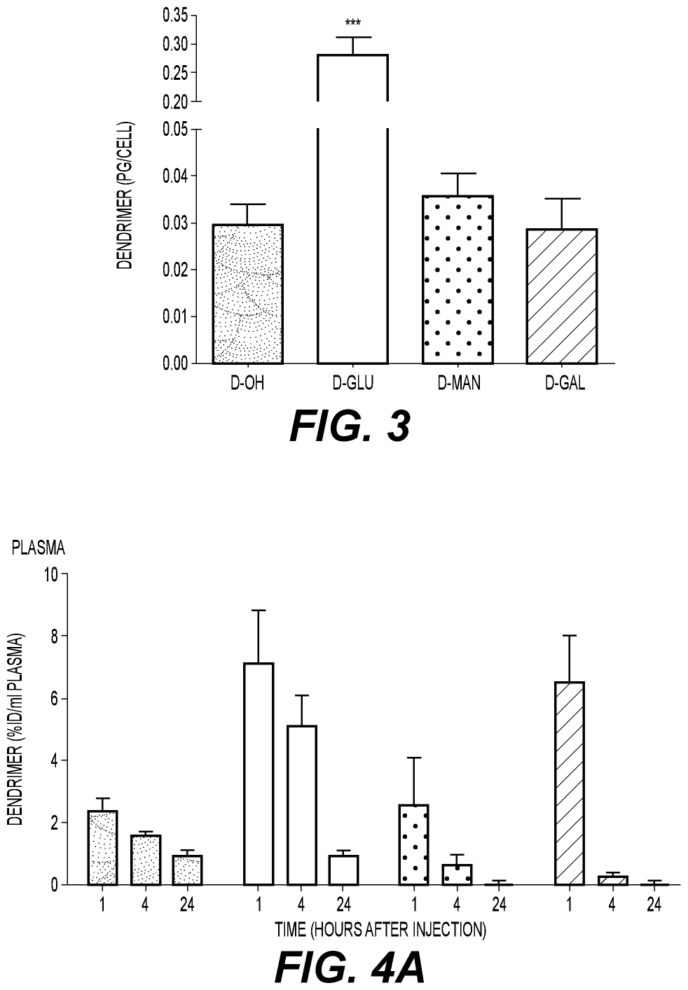

D-GLU exhibited ˜8-fold higher tumor accumulation compared to unmodified dendrimer (FIG. 2A, p<0.001 D-OH vs. D-GLU). At 24 hours after injection after equivalent dose of dendrimers administered, D-GLU exhibited 15.0±4.7 μg/g tissue compared to unmodified D-OH which exhibited 1.9±0.3 μg/g tissue in the tumor. D-GLU exhibited similar tumor accumulation compared to generation 6 unmodified dendrimers, which experience size-dependent longer circulation time due to decreased renal clearance rate. D-MAN and D-GAL altered the kinetics of dendrimer tumor targeting, shifting the peak from 4 hours earlier to 1 hour post-injection (p=0.072 D-OH vs. D-MAN, p=0.0013 D-OH vs. D-GAL). D-MAN and D-GAL exhibited ˜2- and ˜2.5-fold greater tumor accumulation at 1 hour compared to D-OH, but cleared faster from the tumor and exhibited ˜50% lower levels within the tumor after 24 hours compared to D-OH. These trends are similar to in vitro dendrimer internalization into GL261 tumor cells (FIG. 3), with D-GLU exhibiting ˜10-fold greater internalization than the other dendrimers. In the in vitro GL261 tumor cell assay D-GLU exhibited significantly greater uptake into GL261 tumor cells, while D-MAN and D-Glu exhibited similar uptake to unmodified dendrimer D-OH. Notably, all sugar-modified dendrimers significantly improved dendrimer specificity for the brain tumor compared to healthy brain tissue of the contralateral hemisphere (FIG. 2B, p=0.0007 D-OH vs. D-GLU, p=0.0012 D-OH vs. D-MAN, p=0.001 D-OH vs. D-GAL).

D-OH exhibited a tumor/contralateral hemisphere ratio of 3.4±1.0 24 hours after administration, while D-GLU exhibited 18.8±5.4, D-MAN exhibited 4.0±0.4, and D-GAL exhibited 7.1±1.7. Compared to quantification of liposomal nanoparticle tumor targeting in an orthotopic brain tumor model, D-GLU demonstrated ˜100-fold greater tumor accumulation, while D-MAN and D-GAL exhibited ˜8-fold greater tumor accumulation. D-GAL and D-MAN performed similarly to gold and PEGylated iron oxide nanoparticles, while D-GLU exhibited ˜10-fold greater tumor accumulation. Specificity for the tumor of these sugar-modified dendrimers also compared favorably to other nanoparticles. In addition, these D-GLU and D-MAN exhibited highly specific localization within TAMs and activated microglia while D-GAL targeted the tumor extracellular space, whereas quantitative nanoparticle accumulation studies do not explore cell-type localization. Therefore, these dendrimers can be applied for targeted, highly specific tumor targeting. Even though many nanoparticles are known to show a high accumulation into the tumor by the enhanced permeability and retention effect, this does not translate to intracellular accumulation. In addition, the higher TAM intracellular accumulation of glucose-modified dendrimer could enable localized TAM immune programming from systemic administration.

Modification of Dendrimers with Sugars Moderately Alters Systemic Biodistribution

To evaluate impacts on off-target accumulation, organ accumulation of sugar-modified dendrimers to D-OH were also compared to assess systemic biodistribution of sugar-conjugated dendrimers compared to unmodified dendrimers (FIGS. 4A-4F). Glioblastoma bearing mice were injected with unmodified (D-OH), glucose-(D-GLU), mannose-(D-MAN), and galactose-conjugated (D-GAL) dendrimers on day 14 after tumor inoculation.

All dendrimers were rapidly cleared from circulation, with less than 1% of the injected dose per mL plasma remaining in circulation 24 hours after injection (FIG. 4A). D-GLU exhibited similar kidney levels to D-OH, while D-MAN and D-GAL were cleared significantly more rapidly from kidneys than D-OH (FIG. 4B, p<0.0001 D-OH vs. D-MAN, p=0.0004 D-OH vs. D-GAL).

Sugar-dendrimer conjugates did exhibit significantly increased accumulation within the livers compared to D-OH, with D-OH exhibiting 0.29±0.04 μg/g tissue, D-GLU exhibiting 7.8±3.0 μg/g tissue, D-MAN exhibiting 1.7±0.30 μg/g tissue, and D-GAL exhibiting 10.5±2.0 μg/g tissue. Consistent with these findings, glucose transporters with high uptake rates have been found in the liver. Significantly increased liver accumulation was also expected with D-GAL, which targets galactose receptors on hepatocytes for implications in delivery to liver diseases. Notably, despite increased liver accumulation, these sugar-modified dendrimers still exhibit lower liver content compared to free chemotherapies and other nanoparticles with systemic administration. Sugar dendrimers also exhibited altered kinetics in the spleen, with D-MAN accumulating more slowly and D-GAL exhibiting significantly lower spleen accumulation (FIG. 4D, p=0.0143 D-OH vs. D-MAN, p<0.0001 D-OH vs. D-GAL). Notably, D-GLU exhibited significantly greater accumulation in lungs (FIG. 4E, p=0.0002 D-OH vs. D-GLU) and hearts (FIG. 4F, p=0.003 D-OH vs. D-GLU). This is consistent with findings where lungs and hearts uptake glucose due to high energy demands.

### Glucose-Conjugated Dendrimer Increases Cellular Internalization and BBB Penetration

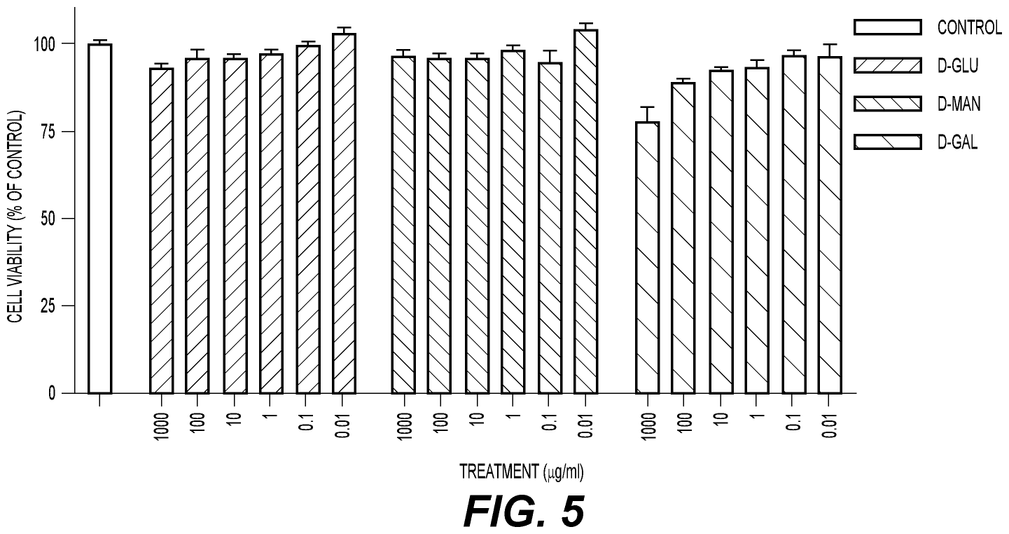

It has been shown that dendrimer surface decoration with mannose moieties alters the dendrimer internalization pathway from fluid-phase endocytosis to mannose receptor-mediated uptake but did not alter the overall magnitude of dendrimer internalization compared to unmodified dendrimers. To confirm that D-GLU similarly interacted with the expected receptor and to explore how the significantly increased tumor accumulation arises, D-GLU uptake in BV2 murine microglia was investigated. To check cytotoxic effects, sugar dendrimers were first assessed for cell viability in microglia in vitro after 24 hours of exposure. Sugar dendrimers did not exhibit any cytotoxicity apart from slight toxicity with D-GAL at high dose, consistent with previous findings where galactose may induce toxicity in brain tissue at high concentrations (FIG. 5).

STF31, an inhibitor of GLUT1, was used to block interactions with glucose. In the presence of STF31, D-GLU exhibited a significant decrease of ˜20% in cellular internalization while D-OH exhibited a slight nonsignificant decrease of ˜5% (FIG. 6A, p=0.0083 D-GLU-STF31 vs. D-GLU+STF31, p=0.13 D-OH-STF31 vs. D-OH+STF31). This ˜20% in internalization may be due to other glucose transporters, which can compensate for STF31 inhibition of GLUT1 to mitigate inhibition of cellular internalization of D-GLU. These results indicate that conjugation of glucose to the dendrimer surface alters the internalization pathway towards glucose transporters. This interaction with glucose transporters significantly increased internalization of dendrimers in both resting and IL4 activated TAMs-like microglia (FIG. 6B, p=0.0003-IL4 D-GLU vs. D-OH, p=0.0004+IL4 D-GLU vs. D-OH). D-OH exhibited a ˜2-fold increase in internalization between resting and TAMs (p=0.016-IL4 D-OH vs. +IL4 D-OH) while D-GLU did not, indicating that D-GLU targets glucose transporters for enhanced internalization in a phenotype-independent manner. This is consistent with previous reports where GLUT1 expression is impacted by pro-inflammatory phenotype but remains unchanged with anti-inflammatory (pro-tumor) activation, while IL4 stimulation induces increased endocytosis. It was previously demonstrated that D-OH internalization is enhanced in pro-inflammatory activated microglia. D-GLU brain accumulation in healthy mice was examined to see if glucose modification impacted dendrimer BBB penetration. In healthy mice, D-GLU exhibited significantly increased brain penetration compared to D-OH consistent with levels observed in the contralateral hemisphere in brain tumor bearing mice (FIG. 6C, p=0.0012 D-GLU healthy vs. D-GLU CH, p=0.143 D-GLU healthy vs. D-OH healthy). This increased penetration could be due to the interactions with glucose receptors on endothelial cells of the blood brain barrier, which transport nutrients into the brain for normal brain functions but warrants further investigation. Taken together, these results suggest that the significantly increased tumor accumulation exhibited by D-GLU is due to interactions with glucose receptors leading to significantly increased cellular internalization within TAMs.

Galactose-Conjugated Dendrimers Interact with Galectins for Tumor Cell Surface Targeting

D-GAL exhibited significantly altered signal pattern within the glioblastoma tumor compared to unmodified and other sugar modified dendrimers. Galectins are highly upregulated on cancer cell membranes and mediated interactions with the tumor extracellular matrix. To determine if D-GAL was enabling interactions with galectins, GL261 murine glioblastoma cells were exposed to D-GAL or D-OH in the presence or absence of α-lactose, a broad inhibitor of galectins. Membrane and cytosolic fractions of these treated GL261 cells were then separated. D-GAL exhibited significantly greater association with cell membranes than D-OH, and this interaction was inhibited in the presence of α-lactose (FIG. 6D, p=0.027 D-GAL-α-lactose vs. D-OH-α-lactose, p=0.0095 D-GAL-α-lactose vs. D-GAL+α-lactose). D-OH exhibited no change in membrane association in the presence or absence of α-lactose. This confirms that conjugation of galactose moieties to the dendrimer surface enables interactions with galectins on glioblastoma cell membranes. Interestingly, D-GAL exhibited no change in the cytosolic fraction compared to D-OH and in the presence or absence of α-lactose, consistent with previous reports of galectins as mediators of extracellular matrix interactions rather than as internalization pathways (FIG. 6E). These results indicate that D-GAL shifts dendrimer targeting away from TAMs and towards glioblastoma cell membranes by interacting with galectins, with implications for intratumor drug delivery, apart from immunotherapies.

### CONCLUSION

Developments in nanotechnology are providing critically needed tumor-specific, intracellular targeted drug delivery strategies to improve patient outcomes in glioblastoma and other cancers. In this study, three dendrimers precisely modified with glucose, mannose, or galactose sugar moieties were tested as targeting ligands using click chemistry. It was demonstrated that by conjugating mannose, glucose, or galactose to the dendrimer, their tumor specificity was significantly increased with systemic administration, and the kinetics of their tumor accumulation was altered. In addition, these sugar moieties conferred receptor-specific interactions. D-GLU exhibited interactions with glucose transporters on TAMs, resulting in significantly increased TAMs specific tumor accumulation. D-GAL exhibited interactions with galactins on the surface of cancer cells, enabling targeting of the tumor microenvironment. Taken together, these results indicate that the dendrimer is an effective, highly versatile drug delivery platform that can be modified with targeting ligands to tailor their receptor interactions that take advantage of the unique tissue biophysics of hydroxyl dendrimers.

Modifications and variations of the present invention will be apparent to those skilled in the art and are intended to come within the scope of the appended claims. All references cited herein are incorporated by reference.

1. A glycosylated dendrimer, comprising
(a) a dendrimer of generation 0-10;
(b) one or more carbohydrate moieties; and
(c) one or more active agents,
wherein the one or more carbohydrate moieties and the one or more active agents are conjugated, optionally via a linker, to terminal groups on the surface of the dendrimer.

2. The glycosylated dendrimer of claim 1, wherein the dendrimer is a poly(amidoamine) (PAMAM) dendrimer.

3. The glycosylated dendrimer of claim 1, wherein the dendrimer is a generation 2, generation 3, generation 4, generation 5, generation 6, generation 7, or generation 8 dendrimer.

4. The glycosylated dendrimer of any one of claim 1, wherein the dendrimer is a hydroxyl (OH)-terminated dendrimer.

5. The glycosylated dendrimer of any one of claim 1, wherein the carbohydrate moiety is conjugated, optionally via a linker, to between about 1% and about 40%, inclusive, of the total number of terminal groups on the dendrimer.

6. The glycosylated dendrimer of any one of claim 1, wherein the carbohydrate moieties are those that can be transported via one or more glucose transporters selected from the group consisting of GLUT1, GLUT2, GLUT3, GLUT4, GLUT5, GLUT6, GLUT7, GLUT8, GLUT9, GLUT10, GLUT11, GLUT12, GLUT13, and GLUT14.

7. The glycosylated dendrimer of claim 1, wherein the carbohydrate moieties are those that can be transported via GLUT1.

8. The glycosylated dendrimer of claim 1, wherein the carbohydrate moieties are oligosaccharides with terminal groups selected from the group consisting of glucose, glucosamine, mannose, fructose, dehydroascorbic acid, urate, and myo-inositol.

9. The glycosylated dendrimer of claim 1, wherein the carbohydrate moieties are monosaccharides selected from the group consisting of glucose, glucosamine, mannose, fructose, dehydroascorbic acid, urate, and myo-inositol.

10. The glycosylated dendrimer of claim 1, wherein the carbohydrate moieties are one or more glucose molecules.

11. The glycosylated dendrimer of claim 1, wherein the carbohydrate moieties are not galactose.

12. The glycosylated dendrimer of claim 1, wherein the one or more active agent(s) is selected from the group consisting of therapeutic agents, prophylactic agents, and diagnostic agents.

13. The glycosylated dendrimer of claim 1, wherein the one or more active agent(s) is selected from the group consisting of a small molecule, an antibody or antigen-binding fragment thereof, a nucleic acid, and a polypeptide.

14. The glycosylated dendrimer of claim 12, wherein the therapeutic agent is selected from the group consisting of anti-cancer agents, immune-modulatory agents, antimicrobial agents, anesthetic agents, anti-oxidant agents, and anti-angiogenic agents.

15. The glycosylated dendrimer of claim 12, wherein the diagnostic agent is selected from the group consisting of fluorescent dyes, near infra-red dyes, SPECT imaging agents, PET imaging agents, and radioisotopes.

16. The glycosylated dendrimer of claim 1, comprising one or more linkers or coupling agents between the dendrimer and the active agent(s), or between the dendrimer and glucose molecule(s).

17. The glycosylated dendrimer of claim 16, wherein the one or more linkers or coupling agents between the dendrimer and the active agent(s), or between the dendrimer and glucose molecule(s) are one or more oligoethylene glycol chains.

18. The glycosylated dendrimer of claim 1, wherein the active agent(s) and glucose molecule(s) are conjugated to the dendrimer via one or more linkages selected from the group consisting of disulfide, ester, ether, thioester, and amide.

19. A pharmaceutical formulation comprising the dendrimer of claim 1 and a pharmaceutically acceptable carrier or excipient.

20. The pharmaceutical formulation of claim 19 wherein the formulation is formulated for intravenous or intraperitoneal administration.

21. The pharmaceutical formulation of claim 19, wherein the formulation is formulated for oral administration.

22. A method for treating or preventing one or more symptoms of a disease and/or disorder in a subject in need thereof, comprising administering to the subject the pharmaceutical formulation of claim 19,
wherein the formulation is administered in an amount effective to treat, alleviate or prevent one or more symptoms of the disease and/or disorder.

23. The method of claim 22, wherein the one or more disease and/or disorder is a proliferative disease.

24. The method of claim 23, wherein the proliferative disease is cancer.

25. The method of claim 24, wherein the cancer is a cancer of the brain and/or CNS selected from the group consisting of gliomas, glioblastoma multiforme, gliosarcoma, astrocytoma, oligodendroglioma, ependymoma or intracranial ependymoblastoma, meningioma, medulloblastoma, ganglioma, head and neck squamous cell carcinoma, Schwannoma, craniopharyngioma, cordomas and pituitary tumor.

26. The method of claim 25, wherein the symptoms of the brain cancer or CNS cancer include one or more selected from the group consisting of headaches, seizures (fits), persistently feeling sick (nausea), being sick (vomiting) and drowsiness, mental or behavioral changes, such as memory problems or changes in personality, progressive weakness or paralysis on one side of the body, and vision or speech problems.

27. The method of claim 22, wherein the amount of active agent effective to treat or prevent the one or more symptoms is less than the amount of the same active agent administered in the absence of the dendrimers, or administered as a formulation in combination with dendrimers in the absence of associated glucose molecules.

28. The method of claim 27, wherein the amount of active agent effective to treat or prevent the one or more symptoms is at least 10-fold less than the amount of the same active agent administered in the absence of the dendrimers, or administered as a formulation in combination with dendrimers in the absence of associated glucose molecules.

29. A pharmaceutical formulation comprising the dendrimer of claim 15, and a pharmaceutically acceptable carrier or excipient.

30. The pharmaceutical formulation of claim 29 wherein the formulation is formulated for intravenous or intraperitoneal administration.

31. The pharmaceutical formulation of claim 29, wherein the formulation is formulated for oral administration.

32. A method for labeling a tumor in a subject, comprising administering to the subject the pharmaceutical formulation of claim 29,
wherein the formulation is administered in an amount effective to label one or more cells associated with the tumor.

33. The method of claim 32, wherein the labeling is used to diagnose or identify a tumor in the subject.

34. The method of claim 33, wherein the labeling is used to monitor or guide chemotherapy and/or surgery.

35. The method of claim 22, wherein the formulation is administered to the subject systemically.

36. The method of claim 35, wherein the formulation is administered via the intravenous or intraperitoneal route.

37. The method of claim 36, wherein the formulation is administered via oral administration.

38. The method of claim 22, wherein the formulation is administered prior to, in conjunction, subsequent to, or in alternation with treatment with one or more additional therapies or procedures.

39. The method of claim 38, wherein the one or more additional procedures include administering one or more therapeutic, prophylactic and/or diagnostic agents or radiation therapy.
